(19) United States
(12) Patent Application Publication
(10) Pub. No.: US 2023/0347038 A1
(43) Pub. Date: Nov. 2, 2023

(54) SURGICAL SITE AIR EVACUATION DEVICE AND METHODS OF USE

(71) Applicant: The Board of Supervisors of Louisiana State University and Agricultural and Mechanical College, Baton Rouge, LA (US)

(72) Inventors: Michael Dunham, New Orleans, LA (US); Rohan R. Walvekar, Metairie, LA (US); Daniel W. Nuss, New Orleans, LA (US); Moises Arriaga, New Orleans, LA (US); Kevin Flynn Hoffeseth, Baton Rouge, LA (US)

(21) Appl. No.: 18/018,514

(22) PCT Filed: Jul. 28, 2021
(86) PCT No.: PCT/US2021/043550

§ 371 Date: Jan. 27, 2023

Related U.S. Application Data
(60) Provisional application No. 63/057,701, filed on Jul. 28, 2020.

(51) Int. Cl. A61M 1/00

(52) U.S. Cl. CPC A61M 1/79; A61M 1/87; A61M 2210/0618

(57) ABSTRACT

The present invention is directed to a surgical evacuation device and associated methods of use. The presently disclosed surgical evacuator is configured for use during aerosol-generating procedures to protect health care workers from exposure to potentially infectious aerosolized particles.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### RELATED APPLICATIONS

This application claims priority to, and the benefit of, U.S. Provisional Application No. 63/057,701, filed on Jul. 28, 2020, the entire contents of which are incorporated herein by reference.

### GOVERNMENT INTERESTS

Not applicable.

### FIELD OF THE INVENTION

The present invention is directed to a surgical evacuation device and associated methods for use thereof. Specifically, the surgical evacuation device disclosed herein is configured to reduce exposure of surgical team members to aerosolized infectious material during a surgical procedure.

### BACKGROUND OF THE INVENTION

COVID-19, caused by the Severe Acute Respiratory Syndrome-2 virus (SARS-Co-2), has become a pandemic of historic proportion. Being a novel virus, essentially all humans are susceptible, and it is highly contagious. Although most will have mild illness and ultimately recover, many will progress to critical illness; globally, COVID mortality rates are estimated at 5.7%. The virus is transmitted primarily through respiratory secretions, and is harbored (and shed) in high quantities in the nose, nasopharynx and oral pharynx, even in asymptomatic patients.

To mitigate global spread of COVID-19 and other infectious agents, the World Health Organization (WHO), Centers for Disease Control and Prevention (CDC), and National Institutes of Health (NIH) have issued guidelines for the general public, including hand-, cough- & sneeze hygiene, social distancing, and self-quarantine, among others. For healthcare workers (HCWs), whose risks are substantially higher, additional recommendations include use of situation-specific PPE, plus environmental controls. Because the virus is concentrated in the upper respiratory tract risk appears higher for HCWs in Otolaryngology, Anesthesia, Ophthalmology, Pulmonology, and other disciplines where there is need for close-proximity contact with the patient, which results in close-range aerosol transmission. Practitioners in these specialties have been among the highest casualties of COVID among HCWs globally. With interventions in the upper airway (e.g., endoscopy, intubation and surgical procedures), the virus is easily aerosolized, further increasing risk of infection among HCWs and support staff members.

In Otolaryngology, many of the upper airway surgeries we perform are classified as Aerosol Generating Procedures (AGPs). Nasal, sinus and skull base surgeries are among the highest AGPs, especially when powered instrumentation such as high-speed drills, shavers and debriders are employed. Even more mundane tools, such as suctions and electrocautery, may contribute to virus aerosolization because of the inherent agitation of secretions and “viral plume” generated with these maneuvers, respectively. In China, the site of the first known outbreak of COVID-19, a single endoscopic pituitary tumor surgery resulted in 14 HCWs infected. Internationally, this has resulted in a drastic decline in upper airway procedures, and authorities have recommended cancellation or postponement of all such elective procedures until further notice. Meanwhile, urgent and emergent conditions still require operative management, e.g., maxillofacial trauma, severe epistaxis, tumors, fulminant infections and others.

### SUMMARY OF THE INVENTION

Disclosed herein is a surgical evacuator configured to remove surgical debris that can be created during a surgical procedure. In various exemplary embodiments, the surgical evacuator comprises a suction port, a body, at least one suction vent, or a combination thereof. In embodiments, the suction port is in fluid communication with the body, and the body is in fluid communication with the at least one suction vent. The surgical evacuator can further comprise a HEPA filtered vacuum system.

In embodiments, the surgical debris comprises surgical plume, surgical fumes, intraoperative secretions, blood, infectious aerosols, or a combination thereof. Certain embodiments of the surgical evacuator comprise an ultraviolet light configured to eradicate microorganisms and viruses.

The surgical evacuator can comprise two suction ports. In embodiments, the suction port comprises a tubular extension extending from an external surface of the body, and the suction port is configured to reversibly attach a tubing such that the suction port is in fluid communication with the tubing. The surgical evacuator can further comprise a valve to selectively control fluid communication between the suction port and the tubing. In embodiments, the suction port comprises a Luer-lock or Luer-taper hub. The suction port can be configured to frictionally attach the tubing thereto.

In various embodiments, the surgical evacuator comprises a medical grade metal, a medical-grade polymer, or a combination thereof. Exemplary Medical grade metals include stainless steel, titanium, tantalum, gold, platinum, palladium, or a combination thereof. In embodiments, the surgical evacuator comprises a silicone elastomer, sterilizable plastic, polytetrafluoroethylene, polyether block amide, polyvinyl chloride, or a combination thereof. The surgical evacuator can be reusable. In alternate embodiments, the surgical evacuator is disposable.

The body of the surgical evacuator can comprise a manifold and the manifold can comprise a plurality of suction vents. In embodiments, the surgical evacuator comprises at least three suction vents. The plurality of suction vents can at least partially surround a surgical field. In embodiments, the surgical evacuator is configured for use in open surgical procedures. The surgical evacuator can be configured for use with procedures that comprise an open wound. The surgical evacuator can be configured for use in a tracheostomy procedure, an otological procedure, a laparotomy, or a combination thereof. In embodiments, the surgical evacuator is configured to be placed over a surgical drape. The surgical evacuator can be configured to reside within an opening of a surgical drape. In embodiments, the surgical field comprises an open wound. The surgical field can comprise an otolaryngolical surgical field or an abdominal surgical field. The surgical evacuator can be configured to reside upon a lateral skull of a patient and surround an ear of the patient. In certain embodiments, the surgical evacuator comprises a C-shaped design. The surgical evacuator comprises a substantially circular or substantially rectangular design.

In embodiments, the surgical evacuator is configured to reside upon the ventral side of a neck of a patient. The surgical evacuator can be configured for use in an open airway surgery. The surgical evacuator can be configured for use in a tracheostomy procedure. In some embodiments, the surgical evacuator comprises an arched rectangular design or an arched elliptical design.

The surgical evacuator can comprise at least two manifolds, at least two suction ports, at least two pluralities of suction vents, or a combination thereof. In one embodiment, the first manifold is in fluid communication with a first suction port and a first plurality of suction vents, and a second manifold is in fluid communication with a second suction port and a second plurality of suction vents. The first manifold and second manifold can be disposed on opposite sides of the rectangular design. In various embodiments, the surgical evacuator is flexible.

The surgical evacuator can be configured for use with nasal procedures, nasopharyngeal procedures, upper aerodigestive procedures, dental procedures, or a combination thereof. The surgical evacuator can be configured for transoral or trans-nasal placement. Exemplary upper aerodigestive procedures include oral cavity procedures, peroral endoscopic procedures, or a combination thereof. The surgical evacuator can be configured for use in nasal surgery, sinus surgery, endonasal skull base surgery, anterior skull base surgery, facial trauma surgery or a combination thereof. In certain embodiments, the body comprises an aspirator, and the at least one suction vent comprises an opening of the aspirator. The surgical evacuator can be configured to conform anatomy of the back of a patient’s nose or a patient’s nasopharynx. In embodiments, the aspirator comprises a substantially conical shape or a substantially cylindrical shape.

The surgical evacuator can comprise a cushion that encircles the opening of the aspirator. In embodiments, the cushion comprises any pliable-or semi-pliable medical-grade material. The cushion can comprise a soft rubber-like material. The cushion can comprise a silicone-coated polyurethane material.

In certain embodiments, the surgical evacuator an inflatable cuff that encircles the opening of the aspirator.

The surgical evacuator can be configured to direct suction toward a patient’s oropharynx. In embodiments, the surgical evacuator is configured for placement in a patient’s oral commissure. The surgical evacuator can be configured for inferior nasopharyngeal placement, superior nasopharyngeal, or a combination thereof. In embodiments, the aspirator comprises a means for avoiding a nasopharyngeal vacuum lock. The means for avoiding a nasopharyngeal vacuum lock can comprise at least one gap, hole, or channel along a perimeter of the aspirator opening. In one embodiment, a cushion or inflatable cuff encircles the opening of the aspirator, and the means for avoiding a nasopharyngeal vacuum lock comprises at least one gap, hole, or channel that is disposed upon or within the cushion or inflatable cuff.

In embodiments, the surgical evacuator comprises an inserter that is configured for use with trans-nasal placement of the evacuator, wherein the inserter is configured to receive and hold the surgical evacuator.

One aspect includes a surgical evacuator comprising a suction port, a manifold, and a plurality of suction vents, wherein the plurality of suction vents at least partially surrounds a surgical field. The surgical port can be in fluid communication with the manifold; the manifold can be in fluid communication with the plurality of vents; and the surgical evacuator can be configured to remove surgical debris during an open surgical procedure. In embodiments, the surgical evacuator comprises at least three suction vents. The surgical evacuator can comprise at least five suction vents. In one embodiment, the surgical evacuator comprises at least six suction vents.

An alternate aspect includes a surgical evacuator comprising a suction port, a body, and at least one suction vent, wherein the body comprises an aspirator, and the at least one suction vent comprises an opening of the aspirator. The suction port can be in fluid communication with the aspirator. In one embodiment, the suction port comprises a tubular extension extending from an external surface of the body, and the suction port is configured to reversibly attach a tubing to permit fluid communication between the suction port and the tubing. The body can be in fluid communication with the at least one suction vent; and the surgical evacuator can be configured to remove surgical debris during a nasal or nasopharyngeal surgical procedure.

In yet another aspect, the invention includes a method of removing aerosolized surgical debris during a surgical procedure. In various embodiments, the method comprises obtaining the surgical evacuator in accordance with any of the embodiments disclosed herein. The method can further comprise placing the surgical evacuator around at least a portion of the surgical field and attaching the suction port to a suction system, wherein the suction system comprises a tubing and a HEPA filtered vacuum system. The method can also include the step of activating the suction system to generate a vacuum within the surgical port, the body, and the at least one suction vent; and permitting aerosolized surgical debris to be drawn into the surgical evacuator serially through the at least one suction vent, the body, and exit through the suction port to the tubing for disposal within the HEPA filtered vacuum system.

Another aspect includes a method of removing aerosolized surgical debris during a surgical procedure. In various embodiments, the method comprises obtaining the surgical evacuator in accordance with any of the embodiments disclosed herein and placing the surgical evacuator around at least a portion of the surgical field. The method can also comprise attaching the suction port to a suction system. In embodiments, the suction system comprises a tubing and a HEPA filtered vacuum system. The method can further comprise activating the suction system to generate a vacuum within the surgical port, the manifold, and the plurality of suction vents. In some embodiments, the method includes permitting aerosolized surgical debris to be drawn into the surgical evacuator serially through the plurality of suction vents, the manifold, and then exit through the suction port to the tubing for disposal within the HEPA filtered vacuum system.

An alternate aspect includes a method of removing aerosolized surgical debris during a surgical procedure, wherein the method comprises obtaining a surgical evacuator that is configured for nasopharyngeal use. In embodiments, the method comprises placing the surgical evacuator within a patient’s inferior nasopharynx or within a patient’s superior nasopharynx. The method can also comprise attaching the suction port to a suction system. In embodiments, the suction system comprises a tubing and a HEPA filtered vacuum system. In certain embodiments, the method comprises activating the suction system to generate a vacuum within the surgical port, the body, and the at least one suction vent. The method can further comprise permitting aerosolized surgical debris to be drawn into the surgical evacuator serially through the at least one suction vent, the body, and exit through the suction port to the tubing for disposal within the HEPA filtered vacuum system. In one embodiment, the method comprises an inserter that is configured to receive and hold the surgical evacuator and evacuator tubing, and the inserter is placed into the nasal cavity. The method can further include loading the surgical evacuator into the inserter. In certain embodiments, the step of placing the surgical evacuator comprises passing the evacuator tubing through the patient’s nasal cavity and into the patient’s oral cavity. The tubing can be brought out through the patient’s mouth and pulling tubing to draw the inserter and the surgical evacuator into a nasal passage of the patient. The method can include further pulling of the tubing to draw the surgical evacuator out of the inserter and into the patient’s nasopharynx. In one embodiment, the method includes verifying placement of the surgical evacuator. Placement of the evacuator can be verified endoscopically.

Yet another aspect includes a method of reducing healthcare provider exposure to infectious aerosolized surgical debris during a surgical procedure. In embodiments, the method comprises obtaining a surgical evacuator in accordance with any of the various embodiments disclosed herein. The method can include placing the surgical evacuator around at least a portion of the surgical field and attaching the suction port to a suction system. In certain embodiments, the suction system comprises a tubing and a HEPA filtered vacuum system. The method can also include the step of activating the suction system to generate a vacuum within the surgical port, the body, and the at least one suction vent and capturing infectious aerosolized surgical debris through the surgical evacuator before the infectious aerosolized surgical debris can leave the surgical field, thereby reducing healthcare provider exposure to the infectious surgical debris.

In another aspect, a method of reducing healthcare provider exposure to infectious aerosolized surgical debris during a surgical procedure comprises obtaining a surgical evacuator configured for nasopharyngeal and placing the surgical evacuator within a patient’s inferior nasopharynx or within a patient’s superior nasopharynx. The method can additionally comprise attaching the suction port to a suction system. In embodiments, the suction system comprises a tubing and a HEPA filtered vacuum system. The method can further comprise activating the suction system to generate a vacuum within the surgical port, the body, and the at least one suction vent and capturing infectious aerosolized surgical debris through the surgical evacuator before the infectious aerosolized surgical debris can leave the surgical field, thereby reducing healthcare provider exposure to the infectious surgical debris. In certain embodiments, the method includes an inserter that is configured to receive and hold the surgical evacuator and evacuator tubing. In such embodiments, the method comprises loading the surgical evacuator into the inserter. The step of placing the surgical evacuator can comprise any one or more of the following passing the evacuator tubing through the patient’s nasal cavity and into the patient’s oral cavity; bringing the tubing out through the patient’s mouth; pulling tubing to draw the inserter and the surgical evacuator into a nasal passage of the patient; and further pulling of the tubing to draw the surgical evacuator out of the inserter and into the patient’s nasopharynx. In one embodiment, placement of the surgical evacuator is verified endoscopically.

Other objects and advantages of this invention will become readily apparent from the ensuing description.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1A shows a top perspective view of a surgical evacuator under one embodiment. The surgical evacuator is shown partially surrounding the ear of a patient.

FIG. 1B provides a front perspective view of the surgical evacuator of the embodiment of FIG. 1A.

FIG. 1C shows a rear perspective view of the surgical evacuator of the FIG. 1A embodiment.

FIG. 2A shows a schematic bottom perspective view of the FIG. 1A embodiment with the body show in phantom.

FIG. 2B shows a top view of one the FIG. 1A embodiment with the body shown in phantom. Non-limiting, exemplary dimensions are shown.

FIG. 2C provides a schematic side view of the FIG. 1A embodiment with the body shown in phantom. Non-limiting, exemplary dimensions are shown in millimeters.

FIG. 3A provides a top perspective view of a surgical evacuator under an embodiment that can be configured for use in open-airway surgeries. The surgical evacuator is shown surrounding the ventral side of a patient’s neck.

FIG. 3B provides a top perspective view of the surgical evacuator of FIG. 3A.

FIG. 4A provides a schematic top perspective view of the surgical evacuator of FIG. 3A.

FIG. 4B provides a top view of the FIG. 4A schematic.

FIG. 4C shows a front view of the FIG. 4A schematic.

FIG. 4D provides a rear view of the FIG. 4A schematic.

FIG. 5A provides non-limiting, exemplary dimensions of the FIG. 4A embodiment in millimeters.

FIG. 5B shows non-limiting, exemplary dimensions of the FIG. 4B embodiment in millimeters.

FIG. 5C shows non-limiting, exemplary dimensions of the FIG. 4C embodiment in millimeters.

FIG. 6A provides a top perspective view of an exemplary surgical evacuator configured for transoral placement in the inferior nasopharynx.

FIG. 6B shows a side view the surgical evacuator of FIG. 6A attached to a tubing and trans-orally placed within the inferior nasopharynx in the context of a lateral airway radiograph.

FIG. 7A shows a schematic side view of the FIG. 6A surgical evacuator with a tubing attached thereto. The evacuator and tubing are shown in phantom. Non-limiting, exemplary dimensions are also shown in mm.

FIG. 7B provides a top view of the FIG. 7A evacuator and tubing. Non-limiting, exemplary dimensions are shown.

FIG. 8A provides a schematic side view of the FIG. 6A evacuator. The evacuator is show in in phantom. Non-limiting, exemplary dimensions are shown in millimeters.

FIG. 8B provides a schematic front view of the FIG. 6A evacuator. The evacuator is shown in in phantom.

FIG. 8C shows a schematic top view of the FIG. 6A evacuator. The evacuator is shown in in phantom. Non-limiting, exemplary dimensions are shown in millimeters.

FIG. 8D provides a top perspective schematic view of the FIG. 6A evacuator. The evacuator is shown in phantom.

FIG. 9A is a top perspective view a surgical evacuator configured for placement in the superior nasopharynx, posterior to the posterior choanae.

FIG. 9B shows a side view the surgical evacuator of FIG. 9A attached to a tubing and trans-orally placed posterior to the posterior choanae in the context of a lateral airway radiograph.

FIG. 10A shows a schematic side view of the FIG. 9A surgical evacuator. The evacuator is shown in phantom, and non-limiting, exemplary dimensions are provided in millimeters.

FIG. 10B provides a schematic side view of the FIG. 10A surgical evacuator with the cushion or cuff exploded off the body of the evacuator. The evacuator is shown in phantom, and non-limiting, exemplary dimensions are provided in millimeters.

FIG. 10C is a front schematic view of the surgical evacuator of FIG. 10A. The evacuator is shown in phantom.

FIG. 10D provides non-limiting, exemplary dimensions of FIG. 10C.

FIG. 11A shows a surgical evacuator and tubing configured for trans-nasal insertion, under one embodiment.

FIG. 11B shows a top, front perspective view of an inserter that is configured for use during insertion of the FIG. 11A evacuator.

FIG. 11C provides a side perspective view of the FIG. 11A evacuator aligned with a channel of the FIG. 11B inserter in preparation for placement of the evacuator therein.

FIG. 11D shows a side view of the FIG. 11A evacuator and the FIG. 11B inserter. The inserter is shown in phantom to reveal the evacuator and tubing residing therein.

FIG. 12A is a schematic sideview of the FIG. 11B inserter. The body of the inserter is shown in phantom, and non-limiting, exemplary dimensions are provided in millimeters.

FIG. 12B provides a schematic top of the FIG. 11B inserter. Non-limiting, exemplary dimensions are provided in millimeters.

FIG. 13A provides a front perspective view of a nasopharyngeal surgical evacuator under one embodiment.

FIG. 13B shows a side view the surgical evacuator of FIG. 13A placed within the nasopharynx in the context of a standard lateral x-ray of the nasopharynx.

FIG. 14 shows a non-limiting example of an evacuator design configured for internal use.

FIG. 15A provides a photographic view of a high-speed photography set up for experimental use in recording simulated surgical aerosols and smoke under one exemplary embodiment.

FIG. 15B provides a photographic view of a high-speed photography set up for experimental use in recording simulated surgical aerosols and smoke under another exemplary embodiment.

FIG. 16A provides a photographic front view of an exemplary cervical manikin equipped with nebulizer to simulate stomal aerosols.

FIG. 16B shows a photographic top perspective view of the FIG. 16A exemplary cervical manikin.

FIG. 17A shows a top perspective view of a surgical site evacuator design under one exemplary embodiment.

FIG. 17B provides a top perspective view of a surgical site evacuator design under another exemplary embodiment.

FIG. 17C shows a top perspective view of a surgical site evacuator design under an additional exemplary embodiment.

FIG. 18 shows a non-limiting example of an electrocautery pencil fitted with a smoke evacuation enclosure.

FIG. 19 shows a non-limiting photographic example of electrocautery tissue simulation on chicken breast tissue. The top tissue provides a view of a non-stimulated chicken breast. The bottom tissue shows a chicken breast following electrocautery stimulation.

FIGS. 20A-20C show side photographic views of effects of site evacuator flow rates under exemplary embodiments at 65 liters per minute (LPM) (FIG. 20A), 86 LPM (FIG. 20B), and 130 LPM (FIG. 20C).

FIGS. 21A-21C show non-limiting examples of effects of site evacuator positioning at inferior (FIG. 21A), oblique (FIG. 21B), and lateral (FIG. 21C) positions. FIGS. 21A & 21B provide side views of the surgical site operator in operation. FIG. 21C provides an overhead view of an embodiment using two surgical site evacuators.

FIGS. 22A-22C show side photographic views of effects of suction vent size on performance of surgical site evacuators in various embodiments. FIG. 22A employs a surgical site evacuator with a suction vent size of 50 mm². FIG. 22B employs a surgical site evacuator with a suction vent size of 150 mm². (FIG. 22C employs a surgical site evacuator with a suction vent size of 70 mm² (FIG. 22C).

FIG. 23A shows an exemplary overhead view of surgical site clearance utilizing a of single surgical site evacuator under one embodiment.

FIG. 23B shows an exemplary overhead view of surgical site clearance utilizing a dual surgical site (panel B) evacuator.

FIGS. 24A-24D show side photographic views of the electrocautery pencil of FIG. 18 in use with different electrocautery power settings. In FIG. 24A, the electrocautery power setting is off. In FIG. 24B, the electrocautery power setting is at 20W. In FIG. 24C, the electrocautery power setting is set to 40W. In FIG. 24D, the electrocautery power setting is 100W.

FIGS. 25A-25C show side photographic views of the electrocautery pencil of FIG. 18 in use in varying operation modes. .In FIG. 25A, the electrocautery mode (frequency) is set to “cut.” In FIG. 25B, the electrocautery mode is set to “coag.” In FIG. 25C the electrocautery mode is set to “blend.”

FIG. 26A shows an overhead photographic view of an experimental surgical field with tissue undergoing cauterization using the electrocautery pencil of FIG. 18, wherein the smoke evacuation enclosure surrounding the electrocautery pencil is activated.

FIG. 26B shows an overhead photographic view of electrocauterization wherein a single suction vent of an exemplary surgical site evacuator is activated. Surgical flume can be seen entering the suction vent of the surgical evacuator on the right side of the image.

FIG. 26C shows an overhead photographic view of electrocauterization wherein two suction vents of an exemplary surgical site evacuator are activated. Surgical flume can be seen entering the suction vents of the surgical evacuator on the left and right side of the surgical site./

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The terms “subject” and “patient” as used herein include all members of the animal kingdom including, but not limited to, mammals, animals (e.g., cats, dogs, horses, swine, etc.) and humans.

### Description of Selected Embodiments

Disclosed herein is a surgical evacuation device configured to prevent or reduce exposure of HCWs to aerosolized infectious material during aerosol-generating procedures. Embodiments of the present invention utilize a suction-evacuation system to remove aerosolized particles, surgical plume, secretions, or other aerosolized particles that can be generated during a medical procedure. The surgical evacuator can be designed for use in any medical procedure wherein aerosols are generated. The surgical evacuator can be configured for use within, on, or around a surgical site of a patient to reduce the risk of infection to HCWs without interfering with the HCW’s view of the surgical site and provides unobstructed access to the surgical site during the procedure. In various embodiments, the surgical evacuator can provide on-going and continuous evacuation of secretions, surgical plume, and aerosolized particles for the duration of the procedure. In embodiments, the surgical evacuation device can be configured to form to the anatomy of the area surrounding or adjacent to the surgical site.

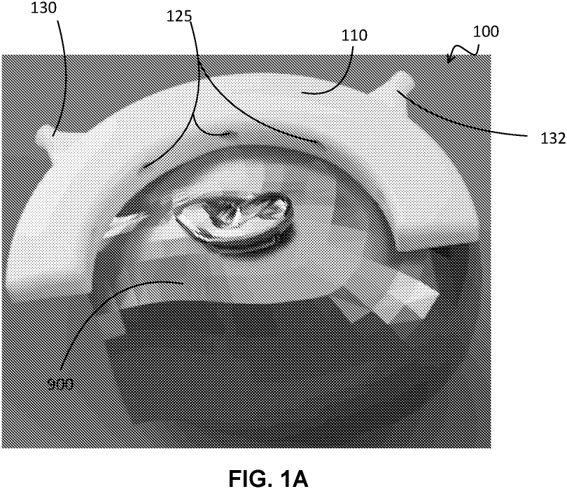

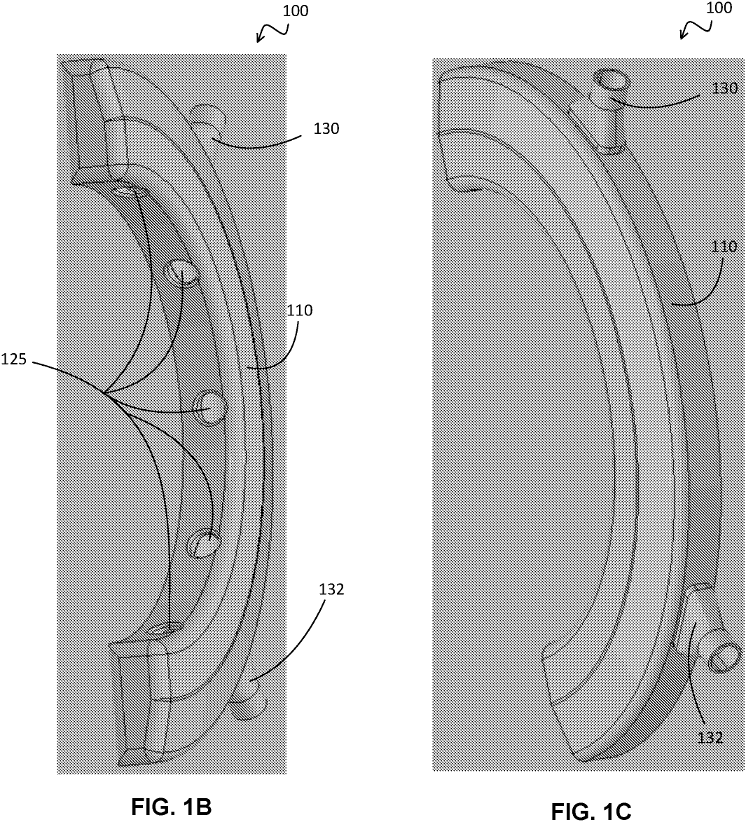

FIG. 1A shows a surgical evacuator 100 that can be configured for use with an open surgical site. The evacuator 100 is shown partially surrounding the ear of a patient 900. In this embodiment, the body 100 of the evacuator is shown lying flat against the lateral skull of the patient 900. The FIG. 1A surgical evacuator 100 can be configured for use with otologic surgical sites. A plurality of suction vents 125 can be seen on a front surface of the surgical evacuator 100 and oriented toward the ear. Two suction ports 130, 132 are visible on a back surface of the evacuator 100.

FIG. 1B provides a front perspective view of the surgical evacuator 100 of the embodiment of FIG. 1A. In this view, the evacuator 100 is rotated about 180° as compared to the FIG. 1A orientation. Once again, a plurality of suction vents 125 is shown on the front surface of the evacuator 100, and two suction ports 130, 132 can be seen extending from the back side of the body 110 of the surgical evacuator 100.

FIG. 1C provides a rear perspective view of the surgical evacuator 100 of the FIG. 1A embodiment. In this figure, the back surface of the surgical evacuator 100 is more clearly shown with the two suction ports 130, 132 extending therefrom.

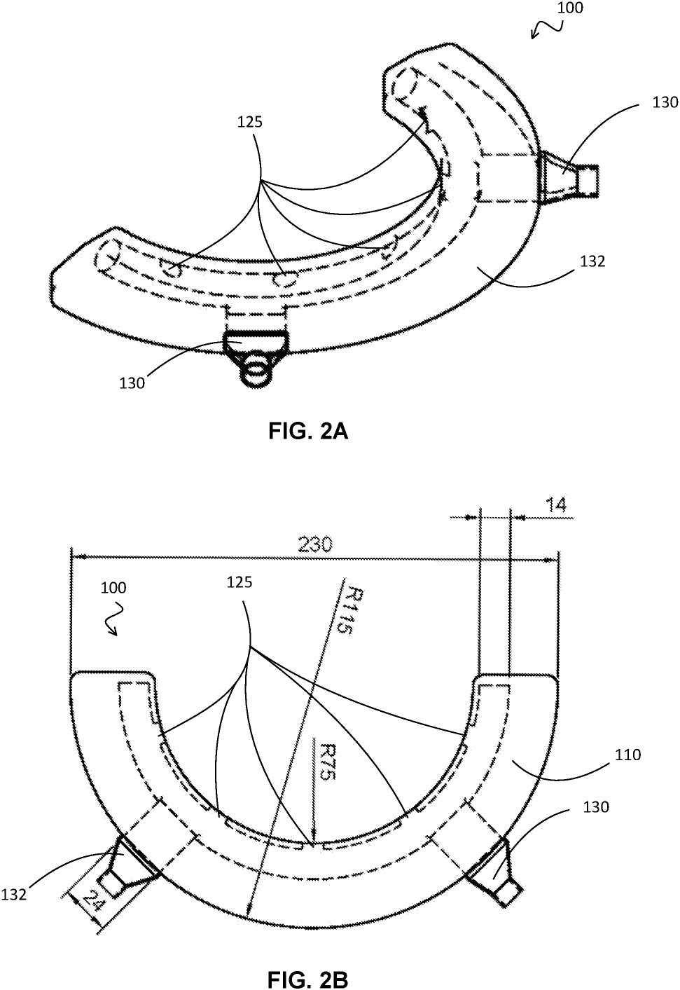

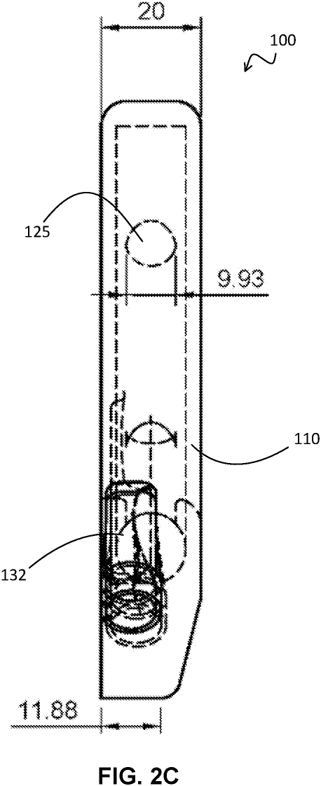

FIG. 2A shows a schematic bottom perspective view of the FIG. 1A embodiment with the body show in phantom to reveal an internal manifold of the evacuator 100. As can be seen in this view, the suction vents 125 are in fluid communication with the manifold, and the manifold is in fluid communication with the suction ports 130, 132.

FIG. 2B shows a top view of one the FIG. 1A embodiment, once again, with the body shown in phantom.

FIG. 2C provides a schematic side view of the FIG. 1A embodiment with the body shown in phantom

The surgical evacuator 100 can be configured to rest up the lateral skull of individuals with a head size between the 5^{th} percentile of females and the 95^{th} percentile of males, inclusive according to any commonly referenced anthropometric standards.¹ In embodiments, the evacuator 100 can be configured to rest upon the lateral skull of an average adult male. The surgical evacuator 100 can be configured to fit the lateral skull of an average adult female. In embodiments, the evacuator 100 is configured to fit the lateral skull of an average-sized 12-year-old child. In certain embodiments, the surgical evacuator 100 is configured to rest upon the lateral skull of an average-sized toddler.

¹A non-limiting example of an acceptable anthropometric standard is Bradtmiller & Friess, A Head-and-Face Anthropometric Survey of U.S. Respirator Users, prepared for National Institute for Occupational Safety and Health (NIOSH) and the National Personal Protective Technology Laboratory, Pittsburgh (May 28, 2004).

In embodiments, the surgical evacuator 100 is up to about 500 mm at its longest point. The longest point of the surgical evacuator 100 can be up to about 300 mm. In certain embodiments, the longest point of the evacuator 100 is up to about 250 mm. The longest point of the surgical evacuator 100 can be less than about 200 mm. In embodiments, the longest point of the surgical evacuator 100 is less than about 100 mm. The longest point of the surgical evacuator 100 can be less than about 50 mm. In embodiments, the longest point of the surgical evacuator 100 is about 50 mm, about 75 mm, about 100 mm, about 125 mm, about 150 mm, about 175 mm, about 200 mm, about 225 mm, about 250 mm, about 275 mm, about 300 mm, about 325 mm, or about 350 mm. In one embodiment, the longest point of the surgical evacuator 100 is about 230 mm.

In certain embodiments, the surgical evacuator 100 the diameter of the manifold is up to about 30 mm. The manifold diameter can be up to about 20 mm. In an embodiment, the manifold comprises a diameter of up to about 15 mm. The diameter of the manifold can be as small as 5 mm. In one embodiment, the manifold diameter is about 10 mm. The manifold diameter can be about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm.

At least one suction port 130, 132 can comprise a diameter of up to about 50 mm. In embodiments, at least one suction port 130, 132 comprises a diameter of less than 10 mm. At least one suction port 130, 132 can comprise a diameter between about 15 mm and about 30 mm. In embodiments, at least one suction port 130, 132 comprises a diameter of about 20 mm, about 21 mm, about 22 mm, about 23 mm, about 24 mm, about 25 mm, about 26 mm, about 27 mm, about 28 mm, bout 29 mm, or about 30 mm.

Although FIGS. 1A-2C show a C-shaped, surgical evacuators 100 configured for use in otologic procedures with alternate shapes and configurations are envisioned. By way of example, the surgical evacuator 100 can comprise a substantially circular embodiment that fully surrounds the surgical site. The evacuator 100 can comprise the shape of a semi-circle. Alternatively, the evacuator 100 can be substantially square or rectangular. In various embodiments, the surgical evacuator 100 comprises an open rectangle or an open square-namely, the evacuator can comprise a three-sided square-like shape or a rectangle-like shape such that the evacuator at least partially surrounds the surgical site.

Although FIGS. 1A-2C show surgical evacuators 100 with 5 suction vents 125, alternate embodiments can have more than 5 suction vents 125. Some embodiments have less than 5 suction vents 125. The surgical evacuator 100 can have more than 10 suction vents 125. In one embodiment, the surgical evacuator 100 comprises up to about 50 suction vents 125. The surgical evacuator 100 can have between 1 and 10 suction vents 125. The evacuator 100 can comprise about 1, about 2, about 3, about 4, about 5, about 6, about 7, about 8, about 9, or about 10 suction vents 125.

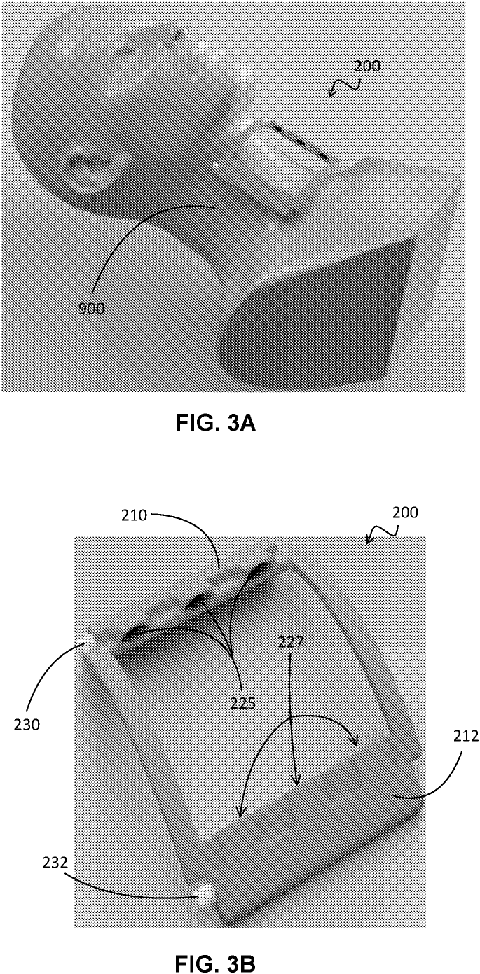

FIG. 3A provides a top perspective view of a surgical evacuator 200 configured for use with an open surgical site in an alternate embodiment. The evacuator 200 is shown surrounding the ventral side of a patient’s 900 neck under one embodiment. This embodiment can be configured for use in open-airway surgeries. As can be seen, the FIG. 3A evacuator 200 can comprise an arched shape that is generally complementary to the shape of a patient’s neck.

FIG. 3B provides a closer, top perspective view of the FIG. 3A surgical evacuator 200. The evacuator 200 in FIG. 3B is rotated 270° as compared to the orientation shown in FIG. 3A. The FIG. 3B evacuator comprises two manifolds 210, 212, and each manifold 210, 212 comprises three suction vents 225, 227. The suction vents 225 of the first manifold 210 are clearly visible in this figure. Each of the manifolds further comprises at least one suction port 230, 232.

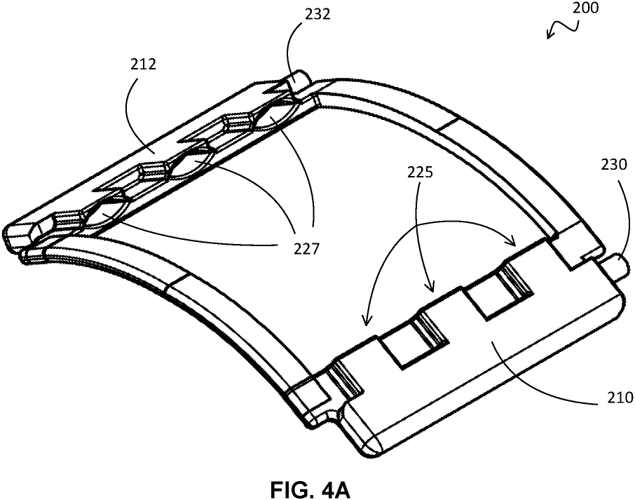

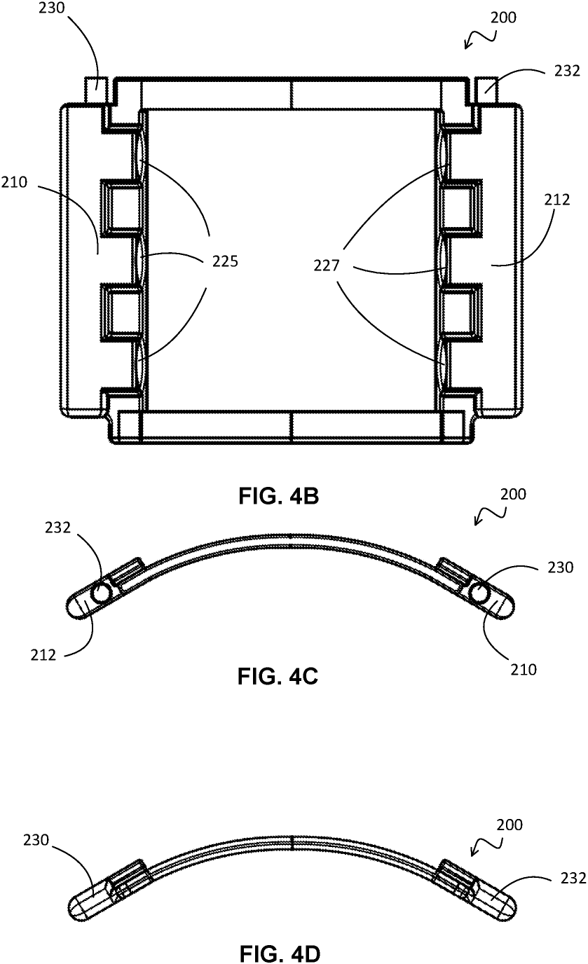

FIG. 4A provides a schematic top perspective view of the FIG. 3A embodiment. The evacuator 200 in the FIG. 4A embodiment is rotated 90° as compared to the orientation shown in FIG. 3A. The suction vents 227 of the second manifold 212 are clearly visible in this orientation.

FIG. 4B provides a top view of the FIG. 4A schematic. In this view, both the first plurality of suction vents 225, and the second plurality of suction vents 227 are visible. This view also more clearly reveals the substantially square shaped opening of the surgical evacuator 200.

FIG. 4C shows a front view of the FIG. 4A schematic. This view looks directly into the suction port 230 associated with the first manifold 210 and the suction port 232 associated with the second manifold 212. FIG. 4D provides a rear view of the FIG. 4A surgical evacuator 200. As can be seen, FIGS. 4C & 4D show the generally arched shape of this embodiment of the surgical evacuator 200, which permits the evacuator 200 to generally conform to the shape of a patient’s neck.

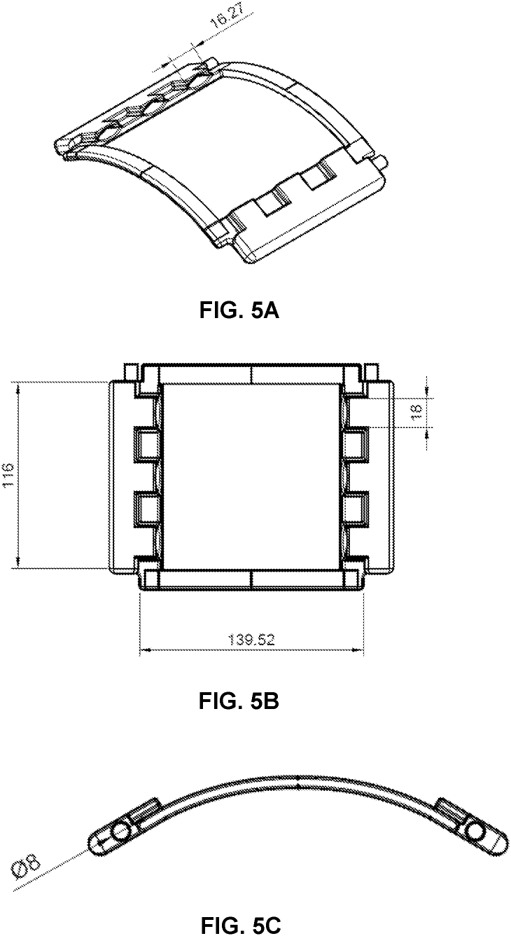

FIGS. 5A-5C provide exemplary dimensions for the embodiments shown in FIGS. 4A-4C. The surgical evacuator 200 can be configured to rest up the ventral neck of individuals with a with a neck size between the 5^{th} percentile of females and the 95^{th} percentile of males, inclusive according to any commonly referenced anthropometric standards. In embodiments, the evacuator 200 can be configured to rest upon the ventral neck of an average adult male. The surgical evacuator 200 can be configured to fit the ventral neck of an average adult female. In embodiments, the evacuator 200 is configured to fit the ventral neck of an average-sized 12-year-old child. In certain embodiments, the surgical evacuator 200 is configured to rest upon the ventral neck of an average-sized toddler. In certain embodiments, the surgical evacuator 200 is configured to fit the dorsal neck of a patient.

The opening of at least one suction vent 225, 227 can be as wide as about 30 mm. In embodiments, the opening of at least one suction vent 225, 227 is as small as about 5 mm. The opening of at least one suction vent 225, 227 can be as wide as about 20 mm. In various embodiments, the opening of at least one suction vent 225, 227 is between about 10 mm and about 20 mm. The opening of at least one suction vent 225, 227 can comprise a width of about 10 mm, about 11 mm, about 12 mm, about 13 mm, about 14 mm, about 15 mm, about 16 mm, about 17 mm, about 18 mm, about 19 mm, or about 20 mm. In one embodiment, the opening of at least one suction vent 225, 227 comprises a width of about 16.5 mm.

In one embodiment, the length along the longitudinal arms of the surgical evacuator 200 are up to about 200 mm. In an embodiment, the longitudinal length of the surgical evacuator 200 is up to about 150 mm. The longitudinal length of the surgical evacuator 200 can be as small as about 50 mm. In embodiments, the longitudinal length of the surgical evacuator 200 is between about 100 mm and 200 mm. The longitudinal length of the surgical evacuator 200 can be about 150 mm. The longitudinal length of the surgical evacuator 200 can be about 110 mm, about 120 mm, about 130 mm, about 140 mm, about 150 mm, about 160 mm, about 170 mm, about 180 mm, about 190 mm, or about 200 mm.

The horizontal length of the surgical evacuator 200 can be up to about 200 mm. In an embodiment, the horizontal length of the surgical evacuator 200 is up to about 150 mm. The horizontal length of the surgical evacuator 200 can be as small as about 30 mm. In embodiments, the horizontal length of the surgical evacuator 200 is between about 100 mm and 150 mm. The horizontal length of the surgical evacuator 200 can be about 120 mm. The horizontal length of the surgical evacuator 200 can be about 50 mm, about 60 mm, about 70 mm, about 80 mm, about 90 mm, about 100 mm, about 110 mm, about 120 mm, about 130 mm, about 140 mm, or about 150 mm. The horizontal length of the surgical evacuator 200 can be about 111 mm, about 112 mm, about 113 mm, about 114 mm, about 115 mm, about 116 mm, about 117 mm, about 118 mm, or about 119 mm.

The diameter of at least one suction port 230, 232 can be up to about 20 mm. In one embodiment, the diameter of the suction port is as small as about 2 mm. The diameter of at least one suction port 230, 232 can be between about 5 mm and about 10 mm, inclusive. In embodiments, the diameter of at least one suction port 230, 232 is about 5 mm, about 6 mm, about 7 mm, about 8 mm, about 9 mm, or about 10 mm.

In the embodiments of FIGS. 3A-5C, each plurality of suction vents 225, 227 is in fluid communication with its respective manifold 210, 212. Each manifold 210, 212 is, in turn, in fluid communication with its respective suction port 230, 232.

In the embodiment of FIGS. 3A-5C, the surgical evacuator 200 comprises a substantially square or a substantially rectangular shape. In alternate embodiments, the evacuator 200 can comprise a substantially circular, elliptical, or oval shape

Although two suction ports 130, 132, 230, 232 are shown in FIGS. 1A-5C surgical evacuators 100, 200, alternate embodiments can comprise more than two suction ports. In embodiments, the surgical evacuator 100, 200 comprise up to 10 suction ports. Embodiments can comprise a single suction port. In various embodiments, the surgical evacuator 100, 200 comprises one, two, three, four, five, six, seven, eight, nine, or ten suction ports.

FIGS. 3A-5C show surgical evacuators 200 with 6 total suction vents 225, 227; however, alternate embodiments can have more than 6 suction vents 225, 227. Some embodiments have less than 6 suction vents 225, 227. The surgical evacuator 200 can have more than 20 suction vents 225, 227. In one embodiment, the surgical evacuator 200 comprises up to about 50 suction vents 125. The surgical evacuator 200 can have a single suction vent 225, 227. The surgical evacuator 200 can have between 2 and 20 suction vents 125. The evacuator 200 can comprise about 2, about 3, about 4, about 5, about 6, about 7, about 8, about 9, about 10, about 11, about 12, about 13, about 14, about 15, about 16, about 17, about 18, about 19, or about 20 suction vents 225, 227.

In various exemplary embodiments, such as that shown in FIGS. 1A-5C, the surgical evacuator can be configured for use in association with an open surgical site. The evacuator can be incorporated into a surgical drape, placed over a surgical drape, or placed underneath a surgical drape. In embodiments, the manifold 110, 210, 212 can comprise a flexible material that is capable of conforming to the area surrounding or within the surgical filed.

Alternate embodiments include surgical evacuators that are configured for use in internal or endoscopic procedures, including, but not limited to aerodigestive surgical procedures. The surgical evacuator can be configured for use in endoscopic sinus surgery, anterior skull base surgery, and facial trauma. Surgical evacuators for internal use can be configured to fit within the oral cavity, the nasopharynx, or a combination thereof. In various embodiments, the surgical evacuator can be configured for introduction to the surgical site via the mouth or nose. The surgical evacuator can be configured to fit within or near the nasopharynx. In embodiments, the evacuator is configured to provide ongoing and continuous collection of blood, secretions, plume, aerosolized surgical debris such as infective particles and organisms, or a combination thereof that may be generated during nasal and sinus procedures. Further, the nasopharyngeal surgical evacuators disclosed herein can be configured to prevent blood, secretions, plume, aerosolized infective particles, or a combination thereof from draining from the nose and back of the nose to the throat of the patient while undergoing a medical procedure, protecting the patient from possible aspiration of these secretions and surgical byproducts.

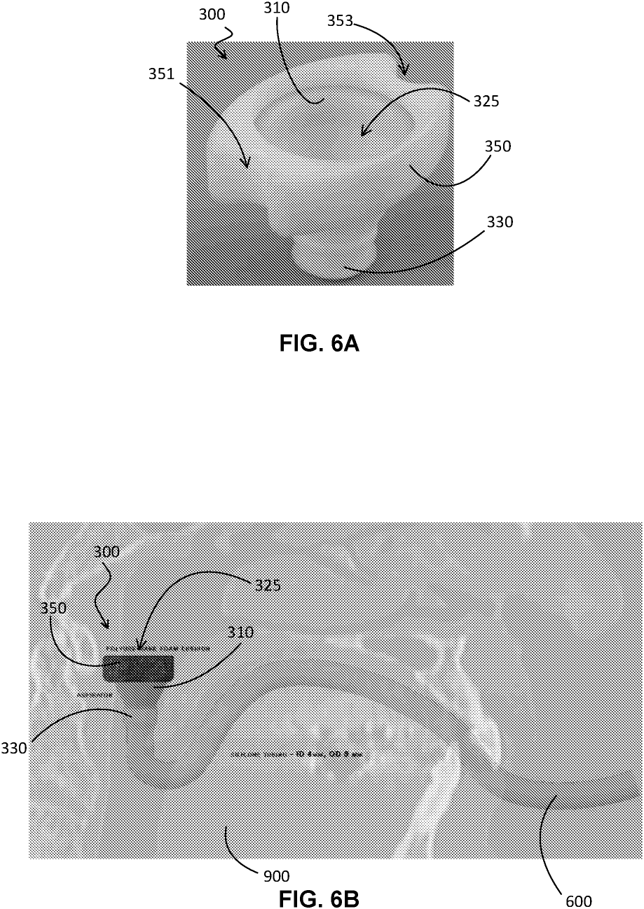

FIG. 6A provides a top perspective view of an exemplary surgical evacuator 300 configured for internal use. Specifically, this embodiment is configured for transoral placement within the inferior nasopharynx. The surgical evacuator 300 comprises a body 310 with an opening that serves as a suction vent 325 for collection of aerosolized particles during a medical procedure. The FIG. 6A surgical evacuator 300 further comprises a suction port 330 that is configured to attach to a tubing (seen at 600 of FIG. 6B). In this embodiment, the body 310 is conical in shape and narrows as the body 310 meets the suction port 330.

The evacuator 300 optionally comprises a cushion 350 or cuff that at least partially surrounds the opening of the body 310. In the FIG. 6A embodiment, the cushion 350 surrounds the opening and protrudes laterally in a generally elliptical or oval shape. The cushion 350 can comprise at least one channel, gap, opening, pass-through, hole, or notch 351, 353. The FIG. 6A embodiment comprises two of such openings 351, 353 that are disposed laterally on opposite sides of the cushion 350. In embodiments, the at least one channel, gap, opening, pass-through, hole, or notch 351, 353 is configured to prevent the formation of a nasopharyngeal vacuum lock in the context or eustachian tube dysfunction.

FIG. 6B shows a side view the surgical evacuator of FIG. 6A attached to a tubing 600 and trans-orally placed within the inferior nasopharynx in the context of a lateral airway radiograph900. As can be seen, the surgical port 330 is in fluid communication with the body 310, which, in turn, is in fluid communication with the suction vent 325. In embodiments, the tubing 600 is attached to a vacuum system, which creates suction such that negative pressure is generated within the surgical evacuator 300 to permit aspiration of the nasopharynx.

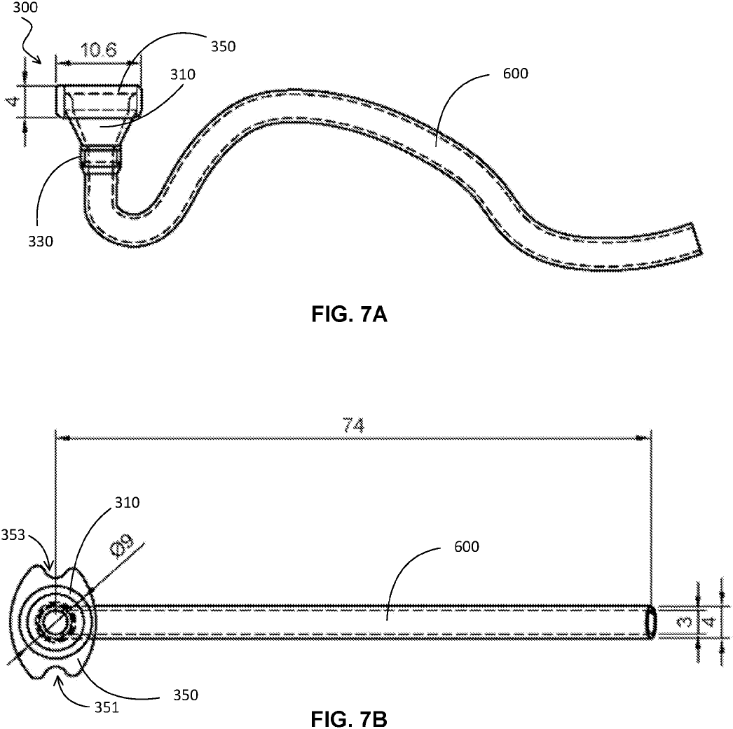

FIG. 7A shows a schematic side view of the FIG. 6A surgical evacuator 300 and FIG. 7B provides a top view of the evacuator 600 with a tubing 600 attached thereto. The evacuator 300 and tubing 600 are shown in phantom to reveal the channel in which negative pressure is established when the tubing is connected to a vacuum system. As can be seen, in this embodiment, the tubing 600 is stretched around the suction port 330 such that it is frictionally secured to the evacuator 300.

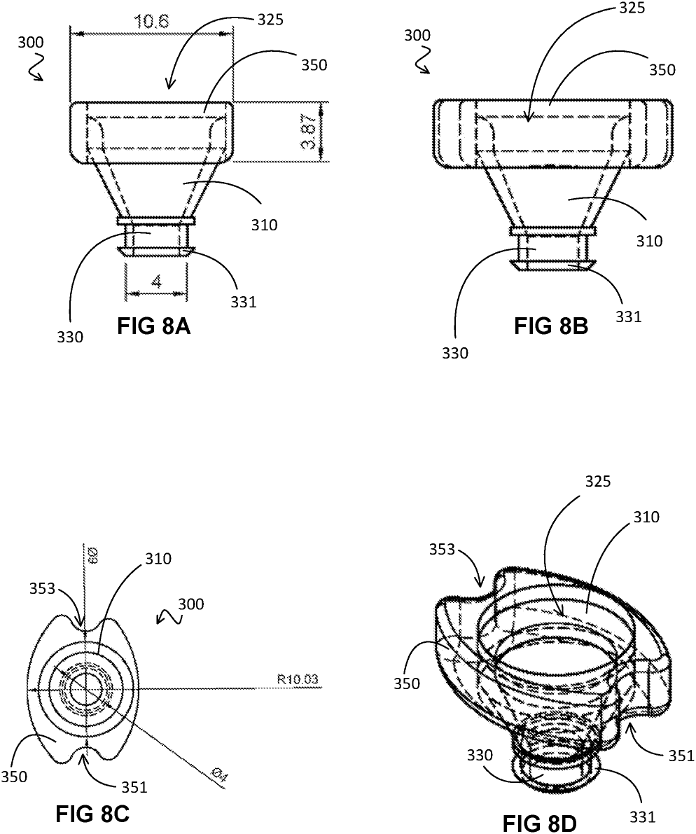

FIG. 8A provides a schematic side view of the FIG. 6A evacuator 300, and FIG. 8B shows a schematic front view of the FIG. 6A evacuator 300. FIG. 8C shows a schematic top view of the FIG. 6A evacuator 300, and FIG. 8D is a top perspective view of the FIG. 6A evacuator. The evacuator 300 of FIGS. 8A-8D is shown in in phantom to reveal the suction vent 325 and internal channel, which serves to collect blood, secretions, plume, aerosolized infective particles and organisms, or a combination thereof. As can be seen, the suction port 330 of the surgical evacuator 300 can comprise a lip 331 that is configured to further secure tubing to the evacuator 300 when attached thereto.

FIGS. 7A-8C provide exemplary dimensions for the FIG. 6A embodiment. The surgical evacuator 300 can be configured to fit within the inferior nasopharynx of individuals with a nasopharynx size between the 5^{th} percentile of females and the 95^{th} percentile of males, inclusive according to any commonly referenced anthropometric standards. In embodiments, the evacuator 300 can be configured to reside within the inferior nasopharynx of an average adult male. The surgical evacuator 300 can be configured to fit the inferior nasopharynx of an average adult female. In embodiments, the evacuator 300 is configured to fit the inferior nasopharynx of an average-sized 12-year-old child. In certain embodiments, the surgical evacuator 300 is configured to reside within the inferior nasopharynx of an average-sized toddler.

The opening of the suction vent 325 can be as wide as about 20 mm. In embodiments, the opening of the suction vent 325 is as small as about 1 mm. The suction vent 325 can comprise an opening that is smaller than 1 mm. In embodiments, the opening of the suction vent 325 can be as wide as about 10 mm. In various embodiments, the opening of the suction vent 325 is between about 1 mm and about 10 mm. The opening of the suction vent 325 can comprise a width of about 1 mm, about 2 mm, about 3 mm, about 4 mm, about 5 mm, about 6 mm, about 7 mm, about 8 mm, about 9 mm, or about 10 mm.

In one embodiment, the cushion 350 can comprise a width of up to about 20 mm. In an embodiment, the cushion 350 is up to about 15 mm wide. The cushion 350 can be as small as about 1 mm wide. In embodiments, the cushion 350 can comprise a length of between about 5 mm and 15 mm. The cushion 350 can comprise a width about 5 mm, about 6 mm, about 7 mm, about 8 mm, about 9 mm, about 10 mm, about 11 mm, about 12 mm, about 13 mm, about 14 mm, or about 15 mm.

In embodiments, the cushion 350 can comprise a height of up to about 15 mm. In an embodiment, the cushion 350 is up to about 10 mm high. The cushion 350 can be as short as about 1 mm. The cushion 350 can be shorter than 1 mm. In embodiments, the cushion 350 can comprise a height of between about 1 mm and 10 mm. The cushion 350 can be as tall as about 1 mm, about 2 mm, about 3 mm, about 4 mm, about 5 mm, about 6 mm, about 7 mm, about 8 mm, about 9 mm, or about 10 mm.

The diameter of the suction port 330 can be up to about 15 mm. In an embodiment, the suction port 330 comprises a diameter of up to about 10 mm. In one embodiment, the diameter of the suction port is as small as about 1 mm. The suction port 330 can comprise a diameter of less than 1 mm. The diameter of the suction port 330 can be between about 1 mm and about 10 mm, inclusive. In embodiments, the diameter of the suction port 330 is about 1 mm, about 2 mm, about 3 mm, about 4 mm, about 5 mm, about 6 mm, about 7 mm, about 8 mm, about 9 mm, or about 10 mm.

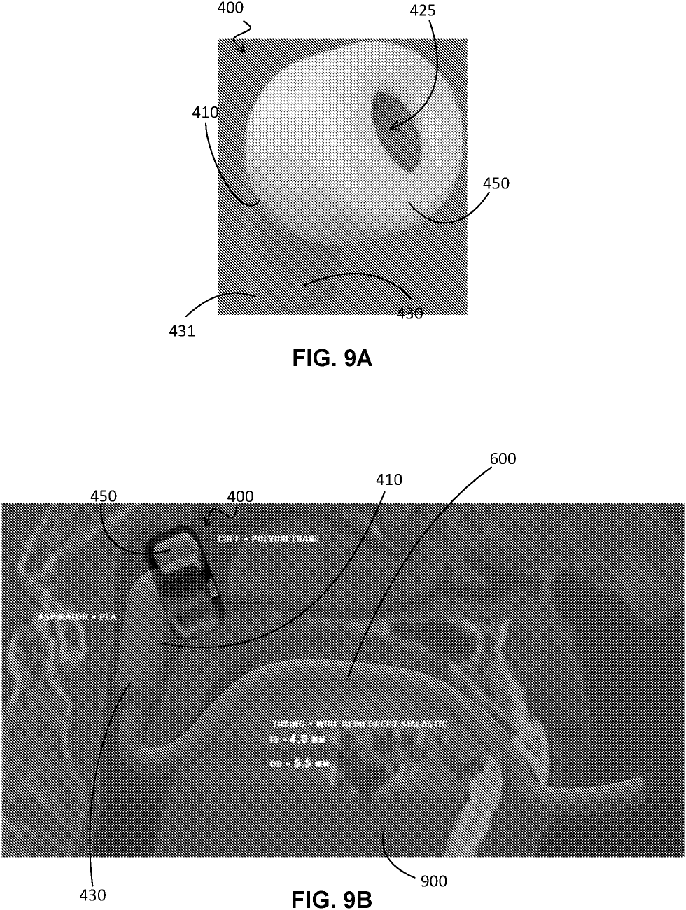

FIG. 9A is a top perspective view of an alternate surgical evacuator 400 configured for internal use. Specifically, this embodiment is configured for placement in the superior nasopharynx, posterior to the posterior choanae. The surgical evacuator 400 comprises a body 410 with an opening that serves as a suction vent 425 for collection of aerosolized particles during a medical procedure. The FIG. 9A surgical evacuator 400 further comprises a suction port 430 that is configured to attach to a tubing (seen at 600 of FIG. 9B). In this embodiment, the body 410 is substantially cylindrical in shape with an angular bend as the body 410 extends toward the suction port 430.

The evacuator 400 can comprise a cushion 450 or cuff that at least partially surrounds the opening of the body 410. In the FIG. 9A embodiment, the cushion 450 encircles the opening in a substantially circular shape. In embodiments, the cushion 450 is configured to permit a full nasopharyngeal seal while evacuating material from the nose.

FIG. 9B shows a side view the surgical evacuator of FIG. 9A attached to a tubing and trans-orally placed posterior to the posterior choanae in the context of a lateral airway radiograph 900. As can be seen, the surgical port 430 is in fluid communication with the body 410, which, in turn, is in fluid communication with the suction vent 425. In embodiments, the tubing 600 is attached to a vacuum system, which creates suction such that negative pressure is generated within the surgical evacuator 400 to permit aspiration of the superior nasopharynx. As can be seen, in this embodiment, the tubing 600 is stretched around the suction port 430 such that it is frictionally secured to the evacuator 400. As can be seen, the suction port 430 of the surgical evacuator 400 can comprise a lip 431 that is configured to further secure tubing 600 to the evacuator 400 when attached thereto.

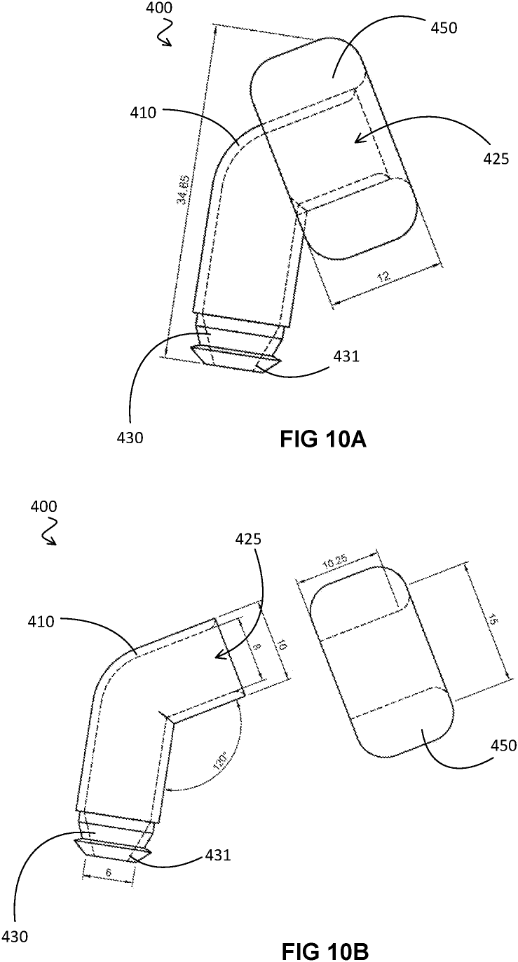

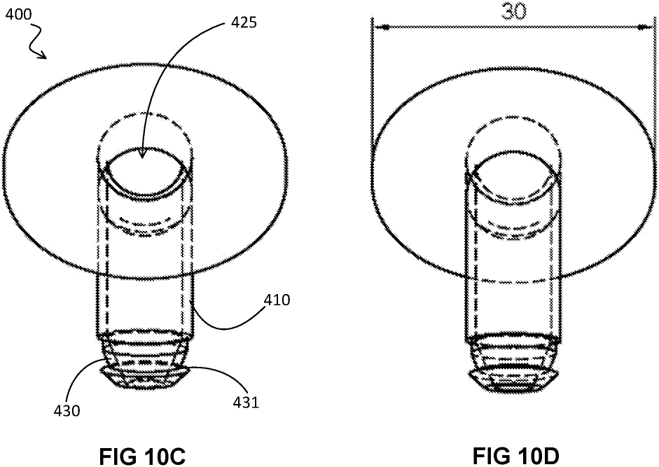

FIG. 10A shows a schematic side view of the FIG. 9A surgical evacuator 400. FIG. 10B provides a schematic side view of the FIG. 10A surgical evacuator with the cushion 450 exploded off the body 410 of the evacuator 400. FIG. 10C is a front schematic view of the surgical evacuator of FIG. 10A.

The evacuator 400 of FIGS. 10A-10D is shown in in phantom to reveal the suction vent 425 and internal channel, which serves to collect blood, secretions, plume, aerosolized infective particles and organisms, or a combination thereof.

FIGS. 10A, 10B, and 10D provide exemplary dimensions for the FIG. 9A embodiment. The surgical evacuator 400 can be configured to fit within the superior nasopharynx of individuals with a with a nasopharynx size between the 5^{th} percentile of females and the 95^{th} percentile of males, inclusive according to any commonly referenced anthropometric standards. In embodiments, the evacuator 400 can be configured to reside within the superior nasopharynx of an average adult male. The surgical evacuator 300 can be configured to fit the superior nasopharynx of an average adult female. In embodiments, the evacuator 400 is configured to fit the superior nasopharynx of an average-sized 12-year-old child. In certain embodiments, the surgical evacuator 400 is configured to reside within the superior nasopharynx of an average-sized toddler.

The opening of the suction vent 425 can be as wide as about 20 mm. In embodiments, the opening of the suction vent 425 is as small as about 1 mm. The suction vent 425 can comprise an opening that is smaller than 1 mm. In embodiments, the opening of the suction vent 425 can be as wide as about 10 mm. In various embodiments, the opening of the suction vent 425 is between about 1 mm and about 10 mm. The opening of the suction vent 425 can comprise a width of about 1 mm, about 2 mm, about 3 mm, about 4 mm, about 5 mm, about 6 mm, about 7 mm, about 8 mm, about 9 mm, or about 10 mm.

In one embodiment, the cushion 450 can comprise a diameter of up to about 50 mm. In an embodiment, the cushion 450 diameter is up to about 40 mm. The diameter of the cushion 450 can be as small as about 10 mm. In embodiments, the cushion 450 can comprise a diameter of between about 25 mm and 35 mm. The cushion 450 can comprise a diameter of about 25 mm, about 26 mm, about 27 mm, about 28 mm, about 29 mm, about 30 mm, about 31 mm, about 32 mm, about 33 mm, about 34 mm, or about 35 mm.

In embodiments, the cushion 450 can comprise a height of up to about 30 mm. In an embodiment, the cushion 450 is up to about 20 mm tall. The cushion 450 can be as short as about 5 mm. The cushion 450 can be shorter than 5 mm. In embodiments, the cushion 450 can comprise a height of between about 10 mm and about 20 mm. The cushion 450 can be as tall as about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm.

The diameter of the suction port 430 can be up to about 20 mm. In an embodiment, the suction port 430 comprises a diameter of up to about 10 mm. In one embodiment, the diameter of the suction port is as small as about 1 mm. The suction port 430 can comprise a diameter of less than 1 mm. The diameter of the suction port 430 can be between about 1 mm and about 10 mm, inclusive. In embodiments, the diameter of the suction port 430 is about 1 mm, about 2 mm, about 3 mm, about 4 mm, about 5 mm, about 6 mm, about 7 mm, about 8 mm, about 9 mm, or about 10 mm.

In various embodiments, the angle of the body 410 can be up to about 150°. The angle of the body 410 can be up to about 130°. In one embodiment, the angle of the body 410 is as small as about 90°. The angle of the body 410 can range from about 115° to about 125°. In embodiments, the angle of the body 410 is about 115°, about 116°, about 117°, about 118°, about 119°, about 120°, about 121°, about 122°, about 123°, about 124°, or about 125°.

The height of the surgical evacuator 400 with a cushion 450 attached thereto can be up to about 50 mm. In embodiments, the height of the evacuator 400 and cushion 450 is up to about 40 mm. The evacuator 400 and cushion 450 can be as short as about 15 mm. In embodiments, the height of the surgical evacuator 400 and cushion 450 range from about 30 mm to about 40 mm. The height of the evacuator 400 and cushion 450 can be about 30 mm, about 31 mm, about 32 mm, about 33 mm, about 34 mm, about 35 mm, about 36 mm, about 37 mm, about 38 mm, about 39 mm, or about 40 mm.

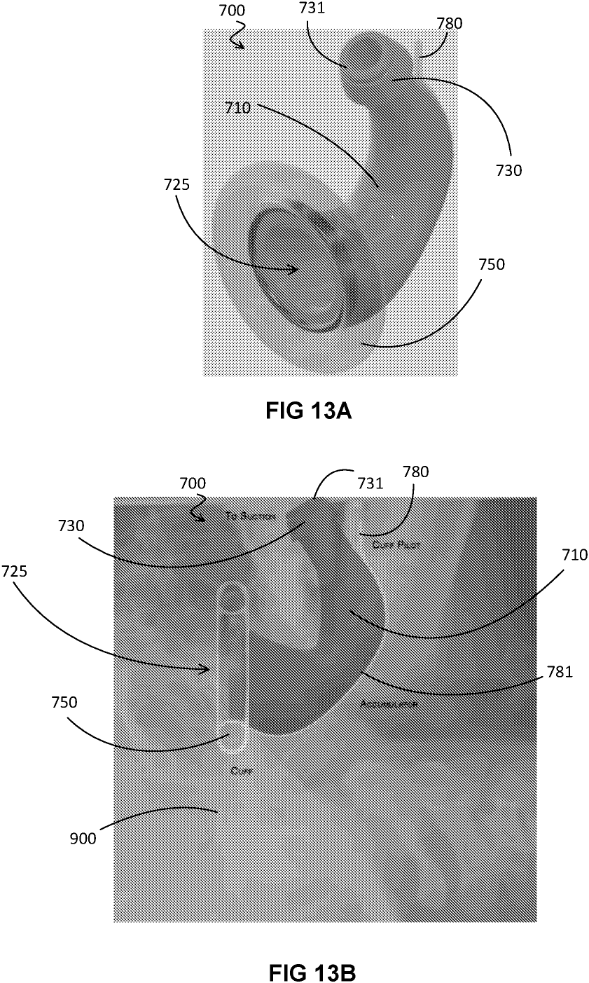

In various embodiments, the cushion 350, 450 can be comprised of silicone-coated polyurethane. The cushion 350, 450 can comprise any material that is suitable for placement within the nasopharynx without injuring the surrounding tissue. In embodiments, the cushion 350, 450 comprises a soft, pliable or semi-pliable material. The cushion 350, 450 can comprise a soft rubber-like material. The cushion 350, 450 can comprise soft rubber. In embodiments, the cushion 350, 450 comprises a medical grade silicone. In certain embodiments, inflatable cuffs (see 750 at FIGS. 13A & 13B for an example) can be used as a substitute for the cushions 350, 450.

In operation of various embodiments, trans-orally inserted evacuators 300, 400 are configured to be pressed into position digitally by lifting the soft palate with the evacuators’ leading edge. The soft cushion 350, 450 around the evacuator 300, 400 can hold the evacuator in place. Further, the shape of the evacuator 300, 400 can permit he surgeon to easily pass the assembly behind the soft palate.

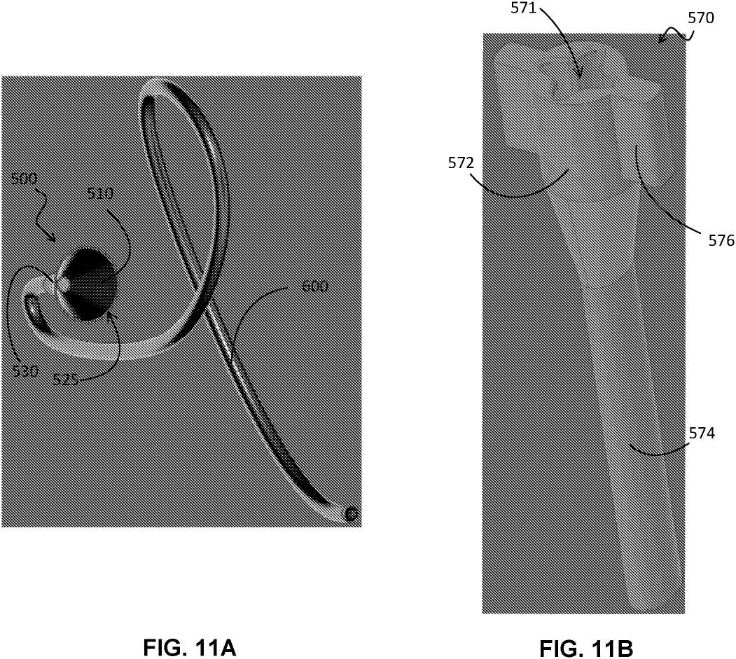

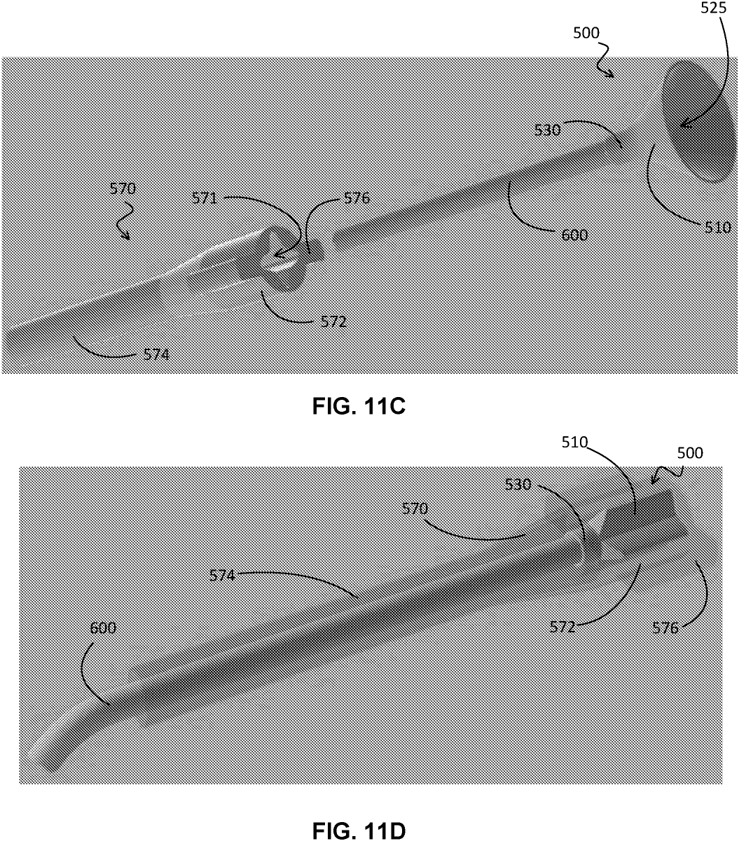

FIG. 11A shows a surgical evacuator 500 under yet another embodiment with tubing 600 attached thereto. The FIG. 11A embodiment can be configured for trans-nasal insertion into the nasopharynx. The surgical evacuator 500 comprises a body 510 with an opening that serves as a suction vent 525 for collection of aerosolized particles during a medical procedure. The FIG. 11A surgical evacuator 500 further comprises a suction port 530 that is configured to attach to the tubing 600. In this embodiment, the body 510 is conical in shape and narrows as the body 510 meets the suction port 530. As can be seen, the surgical port 530 is in fluid communication with the body 510, which, in turn, is in fluid communication with the suction vent 525. In embodiments, the tubing 600 is attached to a vacuum system, which creates suction such that negative pressure is generated within the surgical evacuator 500 to permit aspiration of the nasopharynx.

FIG. 11B shows a top, front perspective view of an inserter 570 that is configured for use during insertion of the FIG. 11A evacuator 500. In embodiments, the inserter 570 is comprises a channel 571 that is configured to receive and hold the surgical evacuator 500 of FIG. 11A and at least a portion of the tubing 600 attached thereto. The inserter 570 comprises a head 572 and an elongated body 574. In embodiments, the head 572 comprises a diameter that is larger than that of the body 574. The head 572 can comprise a tapering neck that serves to transition the head 572 to the body 574. The head 572 can further comprise at least one wing 576 that serves as a stopper to prevent the inserter from being pulled into the nasal cavity during insertion of the surgical evacuator 500. As shown in the FIG. 11B embodiment, the inserter 570 can comprise two wings 576. Certain embodiments can comprise more than two wings 576. The inserter 570 can comprise up to four wings 576.

In various embodiments, the surgical evacuator 500 of FIG. 11A can be comprised of a collapsible material such that the evacuator 500 can be drawn into the inserter 570 and temporarily held therein during insertion of the evacuator 500. As seen in FIG. 11B, the channel 571 can include a substantially plus-shaped opening that is configured to assist with the collapse of the conical evacuator 500 of FIG. 11A.

FIG. 11C provides a side perspective view of the FIG. 11A evacuator 500 and tubing 600 aligned with a channel 571 of the FIG. 11B inserter 570 in preparation for placement of the evacuator 500 therein. As can be seen, the inserter 570 is configured such that the tubing 600 can be inserted into the opening of the channel 571 until it passes completely through the body 574 of the inserter 570. The portion of the tubing 600 exiting the inserter 570 can then be pulled such that the evacuator 500 enters the head 572 of the inserter 570 and becomes collapsed therein. In an embodiment, a plunger can be used with the inserter, wherein depression of the plunger assists with passage of the tubing through a patient’s nasal cavity and into the patient’s oral cavity.

FIG. 11D shows a side view of the evacuator 500 after being positioned within the inserter 570. In this view, the inserter 570 is shown in phantom to reveal the evacuator 500 collapsed within the head 572 of the inserter 570 and the tubing 600 partially residing within the body 574 of the inserter 572. At the bottom left of the schematic, the tubing 600 can be seen exiting from the body 574 of the inserter 570

In operation, the tubing 600 can be initially passed through the nasal cavity and retrieved through the mouth of a patient. The inserter 570 can then be passed at least partially into the nasal cavity. In embodiments with at least one wing 576, once the at least one wing 576 of the inserter 570 reaches the opening of a nostril, the inserter 570 is prevented from moving further into the nasal cavity, and the tubing 600 can be pulled to slide the evacuator 500 through the body 574 of the inserter 570 and eventually exit therefrom to assume its intended location within the nasopharynx. In alternate embodiments, the head 572 of the evacuator 500 is sufficiently wide to prevent passage of the inserter 570 completely into the nasal cavity. Such embodiments do not require wings 576. In embodiments, placement of the evacuator 500 can be verified such as through endoscopically confirming correct deployment. As seen in the FIG. 11A embodiment, certain embodiments of evacuators 500 configured for nasal insertion do not have a cushion or cuff encircling suction vent 525.

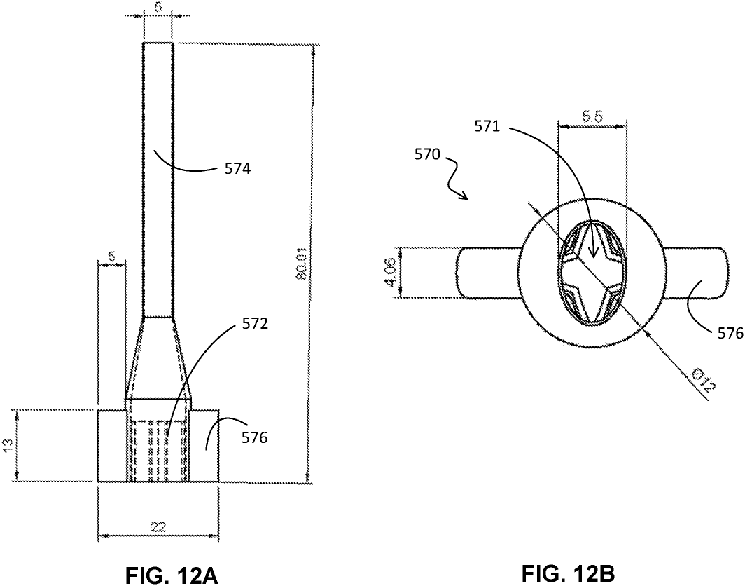

FIG. 12A is a schematic sideview of the FIG. 11B inserter 570, and FIG. 12B provides a schematic top of the inserter 570. Non-limiting, exemplary dimensions of the inserter 570 under one embodiment are provided. The inserter 570 can be up to about 200 mm long. In one embodiment, the inserter 570 is up to 150 mm long. The inserter 570 can be as small as 10 mm long. In certain embodiments, the inserter 570 is between about 10 mm and about 100 mm, inclusive. The inserter 570 can comprise a length of about 10 mm, about 20 mm, about 30 mm, about 40 mm, about 50 m, about 60 mm, about 70 mm, about 80 mm, about 90 mm, or about 100 mm.

The head 572 of the inserter 570 can comprise a diameter as large as 30 mm. In one embodiment, the head 572 of the inserter 570 comprises a diameter of up to 20 mm. The head 572 can comprise a diameter smaller than 10 mm. In certain embodiments, the head 572 of the inserter 570 comprises a diameter between about 10 mm and about 20 mm, inclusive. The head 572 of the inserter 570 can comprise a diameter of about 10 mm, about 11 mm, about 12 mm, about 13 mm, about 14 mm, about 15 mm, about 16 mm, about 17 mm, about 18 mm, about 19 mm, or about 20 mm.

The width of the at least one wing 576 can be up to about 20 mm. In embodiments, the wing 576 can comprise a width less than 1 mm. The wing 576 can be between about 1 mm and about 10 mm wide, inclusive. In various embodiments, the wing 576 comprises a width of about 1 mm, about 2 mm, about 3 mm, about 4 mm, about 5 mm, about 6 mm, about 7 mm, about 8 mm, about 9 mm, or about 10 mm.

The length the at least one wing 576 can be up to about 20 mm. In embodiments, the wing 576 can comprise a length of less than 1 mm. The wing 576 can be between about 1 mm and about 10 mm long, inclusive. In various embodiments, the wing 576 comprises a length of about 1 mm, about 2 mm, about 3 mm, about 4 mm, about 5 mm, about 6 mm, about 7 mm, about 8 mm, about 9 mm, or about 10 mm.

The at least one wing 576 can be up to about 30 mm tall. In embodiments, the wing 576 can comprise a height of less than 5 mm. The wing 576 can be between about 5 mm and about 15 mm tall, inclusive. In various embodiments, the wing 576 comprises a height of about 5 mm, about 6 mm, about 7 mm, about 8 mm, about 9 mm, about 10 mm, about 11 mm, about 12 mm, about 13 mm, about 14 mm, or about 15 mm.

The channel within the body 574 of the inserter 570 comprises a diameter that is sufficient to receive and securely hold commonly used tubing for medical purposes. In certain embodiments, the diameter of the channel within the body 574 of the inserter 570 is up to about 10 mm. The channel within the body 574 of the inserter 570 can comprise of a diameter of less than about 1 mm. In embodiments, the diameter of the channel within the body 574 is between about 1 mm and about 10 mm, inclusive. The diameter the channel within the body 574 can be about 1 mm, about 2 mm, about 3 mm, about 4 mm, about 5 mm, about 6 mm, about 7 mm, about 8 mm, about 9 mm, or about 10 mm.

Surgical evacuators configured for trans-nasal insertion can comprise any medical-grade material. In embodiments, surgical evacuators configured to trans-nasal insertion comprise silicone elastomer, sterilizable plastic, polytetrafluoroethylene, polyether block amide, polyvinyl chloride, or a combination thereof. In certain embodiments, surgical evacuators configured for trans-nasal insertion are comprised of medical grade silicone.

FIG. 13A provides a front perspective view of a nasopharyngeal surgical evacuator 700 under one embodiment that employs an inflatable cuff 750. In this embodiment, the evacuator 700 comprises a substantially J-shaped or C-shaped body with a suction port 730 disposed at one end and a suction vent 725 at the opposite end. The suction port 730 can comprise a lip 731 that is configured to further secure tubing 600 to the evacuator 700 when attached thereto. As can be seen, the surgical port 730 is in fluid communication with the body 710, which, in turn, is in fluid communication with the suction vent 725.

FIG. 13B shows a side view the surgical evacuator 700 of FIG. 13A placed within the nasopharynx in the context of a lateral x-ray of the nasopharynx.900.

The FIG. 13 evacuator 700 comprises an inflatable cuff 750 that at least partially surrounds the opening of the body 710. As shown in this embodiment, the cuff 750 encircles the opening in a substantially circular shape. In embodiments, the cuff 750 can be deflated during insertion. Following proper positioning of the evacuator 700 the cuff 750 can be inflated via the cuff inflation line 780 to the desired level. In embodiments, the cuff can be inflated to permit a full nasopharyngeal seal during a surgical procedure. Following procedure, the cuff 750 can be deflated to permit easier removal of the evacuator 700.

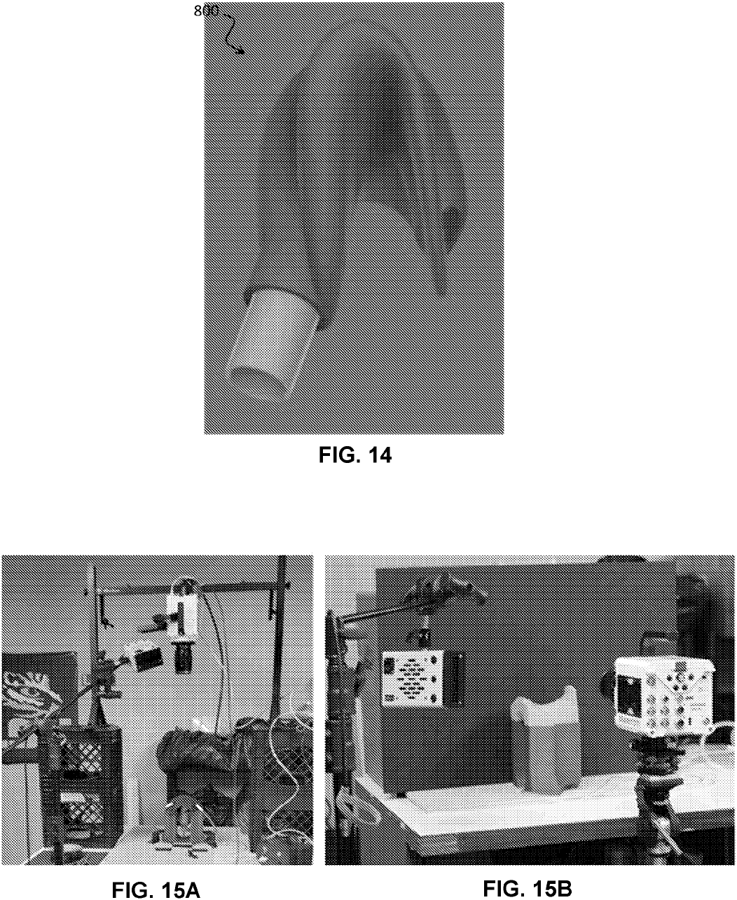

FIG. 14 shows a side view of a surgical evacuator 800 under another embodiment configured for internal use.

Although FIG. 6A-14 show evacuator designs that fit within the nasopharynx, alternate embodiments can be configured for placement in the oral commissure. In addition, embodiments can be configured to direct suction toward the oropharynx.

In the various embodiments disclosed herein, the tubing 600 can comprise a diameter of up to about 15 mm. In an embodiment, the tubing 600 comprises a diameter of up to about 10 mm. In one embodiment, the diameter of the tubing 600 is as small as about 1 mm. The tubing 600 can comprise a diameter of less than 1 mm. The diameter of the tubing 600 can be between about 1 mm and about 10 mm, inclusive. In embodiments, the diameter of the tubing 600 is about 1 mm, about 2 mm, about 3 mm, about 4 mm, about 5 mm, about 6 mm, about 7 mm, about 8 mm, about 9 mm, or about 10 mm. In embodiments, the tubing 600 comprises silicone. The tubing 600 can comprise reinforced silicone tubing.

FIGS. 17A-17C provide different configurations of surgical evacuators in embodiments with a single suction vent. As can be seen, the surgical evacuator 1100 of FIG. 17A comprises a substantially circular suction vent 1125. In the FIG. 17A embodiment, the surgical evacuator 1100 comprises a body 1110 with a cross-sectional shape that is substantially similar to that of the surgical vent 1125. In addition, the suction port 1130 can comprise a cross-sectional shape that is substantially similar to that of the body 1110, the suction vent 1125, or a combination thereof. The surgical evacuator 1200 of FIG. 17B comprises a substantially rectangular suction vent 1225, and the surgical evacuator 1300 of FIG. 17C comprises a substantially rectangular suction vent 1325 with a trough 1310 that extends at least partially through an internal surface of the body 1310. As can be seen in FIGS. 17B and 17C, the surgical evacuator 1200, 1300 can comprise a body 1210, 1310 that tapers from the surgical vent 1225, 1325 to the suction port 1230, 1330. Further, the surgical evacuator 1100, 1200, 1300 can comprise a base 1111, 1211, 1311 that can be utilized to support the surgical evacuator 1100, 1200, 1300 when resting on the skin of the patient during use. In embodiments, the base 1111, 1211, 1311 can be reversibly secured to an area surrounding the surgical field during use. In certain embodiment, an adhesive agent can be applied to an undersurface of the base to further secure the surgical evacuator 1100, 1200, 1300 in place during a surgical procedure. Tape can be applied to the base to reversibly secure the surgical evacuator 1100, 1200, 1300 in place during a surgical procedure. The base 1111, 1211, 1311 can be strapped or tied to the patient. In one embodiment, the base 1111, 1211, 1311 of surgical evacuator 1100, 1200, 1300 can be sutured to the patient. In such embodiments, the base 1111, 1211, 1311 can comprise a hole, eye, notch, gap, or alternate anchor point to which suture may be tied or otherwise secured thereupon.

In certain embodiments, more than one single-vent surgical evacuator 1100, 1200, 1300 can be used in tandem to at least partially surround a surgical field during use. In embodiments, single-vent surgical evacuators can be place on opposite sides of a surgical field (see, for example, FIGS. 21C, 21C, 23B, and 26C). In embodiments, up to twenty single-vent surgical evacuators can be placed around a surgical field. One, two, three, four, five, six, seven, eight, nine, ten, eleven, twelve, thirteen, fourteen, or fifteen single-vent surgical evacuators can be placed around a surgical field.

In various embodiments, one or more suction vents can comprise an opening of between about 10 mm² and 300 mm², inclusive. The suction vent opening can be between about 25 mm² and about 200 mm², inclusive. In certain embodiments, the opening of at least one suction vent can be between about 30 mm² and 175 mm², inclusive. The opening of at least one suction vent can be between about 50 mm² and 150 mm², inclusive The opening of a ate least once suction vent can be less than 10 mm². In certain embodiments, the opening of at least one suction vent is larger than about 300 mm². The opening of at least one suction vent can be about 10 mm², about 20 mm², about 30 mm², about 40 mm², about 50 mm², about 60 mm², about 70 mm², about 80 mm², about 90 mm², about 100 mm², about 110 mm², about 120 mm², about 130 mm², about 140 mm², or about 150 mm².

The surgical evacuator can include a means for neutralizing or eliminating live infectious particles during a medical procedure. In embodiments, the surgical evacuator can be equipped with one or more ultraviolet lights to reduce or eliminate live or infectious particles within and around the surgical site. The ultraviolet light can be configured to reduce or eliminate the presence of live or infectious particles within surgical plume, surgical vapors, aerosolized particles, secretions, or blood.

The evacuators disclosed in various exemplary embodiments can be configured for use in a variety of medical procedures. In embodiments, the evacuators are configured for use in open airway procedures, endoscopic sinus surgery, anterior skull base surgery and facial trauma, oral cavity procedures, peroral endoscopic procedures, neurosurgical procedures, otological procedures, laparotomies, or a combination thereof. The evacuators can be configured for use in nasal procedures, nasopharyngeal procedures, upper aerodigestive procedures, or a combination thereof. In certain embodiments, the surgical evacuators are configured for use during general anesthesia. The evacuators can be configured for use during surgical manipulation of the temporal bone. The evacuators can be configured for use in dental procedures. In embodiments, the evacuators are configured for use in oral surgery.

In the various exemplary embodiments, the surgical evacuator can be configured for use with a HEPA filtered vacuum system that is capable of clearing airborne bio-pathogens, including viruses.

Another aspect of the present invention includes a method of using the surgical evacuators in accordance with any embodiment disclosed within this specification or otherwise apparent from the descriptions herein. In embodiments, an evacuator is placed around a surgical field, and the suction port of the evacuator is attached to a suction system such as a filtered vacuum system. The surgical evacuator can be placed over a surgical drape, within a surgical drape, underneath a surgical drape, or a combination thereof. The suction system can then be activated to being aspiration of the surgical site such that blood, secretions, plume, aerosolized infective particles and organisms, or a combination thereof are removed from the surgical site. In embodiments, the surgical evacuator is employed during the entire surgical procedure. In alternate embodiments, the evacuator is employed during aerosol generating events such as drilling or sawing of bone or other tissue, suctioning, electrocautery, or use of any powered instrumentation during a medical procedure.

In embodiments, the surgical evacuator can be placed at various orientations around the surgical field. In certain embodiments, the surgical evacuator can be placed at an elevated level with respect to the stoma or surgical field. The surgical evacuator can be placed on the same plane as the stoma or surgical field. The surgical evacuator can be placed at a level that is below the stoma or surgical field.

During use of any one or more of the various embodiments disclosed herein, the distance between the opening of the suction vent to the center of the surgical field can vary depending on the needs or preferences of the surgeon, patient, caretakers, or a combination thereof. In embodiments, the suction vent of the surgical evacuator is placed at between about 1 cm away from the center of the stoma or surgical field up to about 30 cm away from the center of the stoma or surgical field. The surgical evacuator can be placed more than 30 cm away from the center of the stoma or surgical field. The surgical evacuator can be placed less than 1 cm away from the center of the stoma or surgical field. In embodiments, the surgical evacuator can be placed such that the distance from the center of the stoma or surgical field to the opening of at least one surgical vent is about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, or about 15 cm.

In certain embodiments, the method of using the surgical evacuator comprises placing the surgical evacuator into the nasopharynx of a patient prior to a surgical procedure. In one embodiment, placement of the surgical evacuator comprises oral introduction of the surgical evacuator. In such embodiments, the evacuator can be digitally pressed into position such as through lifting the soft palate of the patient. In one embodiment, the soft palate can be lifted with the leading edge of the evacuator. The cushion of the device can then be arranged such that the surgical evacuator is held in place during a surgical procedure. In one embodiment, the surgical evacuator is placed in the inferior nasopharynx. The evacuator can be placed in the superior nasopharynx. In an embodiment, the surgical evacuator is placed posterior to the posterior choanae

Alternatively, the surgical evacuator can be introduced through the nasal cavity of a patient. In one embodiment, tubing is passed into the nasal cavity and retrieved through the mouth of the patient. The tubing can then be pulled from the mouth until the evacuator attached thereto enters the nasal cavity and assumes the appropriate position within the nasopharynx.

In one embodiment, and inserter is used to assist with deployment of the evacuator. In such embodiments, the tubing is first attached to the surgical evacuator. The tubing can then be first passed through the inserter and pulled until the evacuator is disposed within the head of the inserter. The tubing is then passed through the nasal cavity and retrieved through the mouth of a patient. The inserter can then be passed at least partially into the nasal cavity until either the head of the inserter or a wing of the inserter reaches the opening of a nostril. The tubing can be further pulled to slide the evacuator through the body of the inserter and eventually exit therefrom to assume its intended location within the nasopharynx. In embodiments, placement of the evacuator can be verified such as through endoscopically confirming correct deployment. After successful evacuator placement, the vacuum system can be engaged to being aspiration through the surgical evacuator. In embodiments, the aspiration continues for the entire surgery. Alternatively, the aspiration can be selective engaged during aerosol-generating tasks.

Also disclosed is a kit that includes a surgical evacuator in accordance with any embodiment disclosed within this specification or otherwise apparent from the descriptions herein. In embodiments, the kit comprises a surgical evacuator and instructions for use thereof. The instructions can be physically provided with the kit or accessible separately from the kit, such as via the retailer’s or manufacturer’s website. The kit can include a tubing for use with the evacuator. In embodiments, the kit includes an ultraviolet light source that is configured to kill viruses, bacteria, or other organisms that can be present in blood, secretions, surgical plume, or other aerosolized particles that can be generated during a surgical procedure. The kit can also include a HEPA filtered vacuum system of HEPA filters capable of clearing airborne bio-pathogens, including viruses. Certain kit embodiments comprise an inline, disposable HEPA filter.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

The technology involves the use of a suction -evacuation system that conforms to the anatomy of the ear and lateral skull that can be incorporated into a surgical drape or placed over the drape. This device collects plume, dust from surgical drilling of the temporal bone, and/or aerosolized particles that may be generated during ear surgical procedures. The device can be used in a patient who is undergoing a procedure under general anesthesia to reduce risk of infection to the surgeon and operative staff from infectious particles and plume that may be generated during surgical manipulation of the temporal bone. In addition, the technology also incorporates the ability to project an ultraviolet light to help eliminate live vial or infectious particles in surgical plume or vapors.

Background and Commercial Applications of the Technology: Surgery of the ear in a patient with COVID-19 poses a high risk of infection; so, a method to minimize transmission risk in such otologic surgeries is valuable. COVID 19 is a highly infectious disease transmitted by droplets and aerosolized virus of patients infected by the Severe Acute Respiratory Syndrome-2 virus (SARS-Co-2)¹ The virus is transmitted primarily through respiratory secretions² in the nose, nasopharynx and oral pharynx, even in asymptomatic patients. Otologic surgery is a particular concern for infection transmission because the Eustachian tube, middle ear and mastoid air cell system are in direct communication with the nose and nasopharynx. Viral studies have confirmed the presence of respiratory corona virus in the middle ear and mastoid.^{3,} ^{4, 5, 6, 7}

Otologic surgery is classified as an Aerosol Generating Procedures (AGPs) due to the high risk of droplet and aerosol generation. Detailed droplet studies have demonstrated that mastoid surgery has an extremely high incidence of droplet contamination of the entire operative team.^{8,} ^{9,} ¹⁰ In addition, aerosols, micron sized debris and infective virus suspended in the air, can remain in the operating room for hours after creation of a viral plume by the powered instrumentation with drills and the suctioning of secretions, irrigation fluid, blood and mucus membranes necessary in ear surgery.

Preoperative testing, personal protective equipment and negative-pressure operating rooms have been recommended to lessen the risk of viral transmission; however, a device to manage droplets and aerosols at the site of generation is particularly important to lessen the operative team’s exposure to infectious droplets and aerosols generated by otologic surgery.

We propose a unique device to help further reduce the risk of droplet and aerosol exposure during otologic surgery. The Continuous Otologic Surgery Site Aspirator is a simple device designed to collect, aspirate and dispose of intraoperative secretions, blood and aerosols positioned at the operative field. The presently disclosed device is capable of providing on-going continuous evacuation of secretions and/or plume and aerosolized infectious particles during otologic procedures while allowing the surgeon to have unobstructed access to the surgical site. Our novel concept of placing this device that conforms to otologic surgical positioning and allows for not only removal of plume, incorporation into existing surgical draping principles but also has the in-built ultraviolet light projector that will help eliminate live infectious particles in the aerosol.

References Cited in This Example:
1. Baud et al, Lancet, March 2020.
2. Centers for Disease Control and Prevention: www.cdc.gov/coronavirus/2019-ncov/hcp/guidance-risk-assesment-hcp.html)
3. Pitkäranta A, Jero J, Arruda E, Virolainen A, Hayden FG. Polymerase chain reaction-based detection of rhinovirus, respiratory syncytial virus, and coronavirus in otitis media with effusion. J Pediatr. 1998 Sep;133(3):390-4.
4. Pitkäranta A, Virolainen A, Jero J, Arruda E, Hayden FG. Detection of rhinovirus, respiratory syncytial virus, and coronavirus infections in acute otitis media by reverse transcriptase polymerase chain reaction. Pediatrics. 1998 Aug; 102(2 Pt 1):291-5.
5. Wiertsema SP, Chidlow GR, Kirkham LA, Corscadden KJ, Mowe EN, Vijayasekaran S, Coates HL, Harnett GB, Richmond PC. High detection rates of nucleic acids of a wide range of respiratory viruses in the nasopharynx and the middle ear of children with a history of recurrent acute otitis media. J Med Virol. 2011 Nov;83(11):2008-17. doi: 10.1002/jmv.22221.
6. Nokso-Koivisto J, Räty R, Blomqvist S, Kleemola M, Syrjänen R, Pitkäranta A, Kilpi T, Hovi T. Presence of specific viruses in the middle ear fluids and respiratory secretions of young children with acute otitis media. J Med Virol. 2004 Feb;72(2):241-8.
7. Heikkinen T, Thint M, Chonmaitree T. Prevalence of various respiratory viruses in the middle ear during acute otitis media. N Engl J Med. 1999 Jan 28;340(4):260-4
8. Shrestha B, Amatya R. Risk of blood splashes in otorhinolaryngology surgery: do we really require protection? Int J Sci Rep. 2017 Jan;3(1):609
9. Lakhani R, Loh Y, Zhang T et al. A prospective study of blood splatter in ENT. Eur Arch Otorhinolaryngol (2015) 272:1809-1812.
10. Shrestha B, Dhakal A, Karmacharya S. Blood splashes risk during otorhinolaryngology surgery: A tertiary care hospital based study. Kathmandu Univ Med J. 2018;64(4):301-5.

### Example 2

This example centers around having a device that is able to provide continuous suction and evacuation of cautery plume during the course of open surgery. The device can also include an ultraviolet light projection feature that be targeted at neutralizing aerosolized infectious particles (e.g. COVID-19)

The Open Surgical Site Air Evacuator can be placed around the surgical site and field. It is particularly intended for use during aerosol generating procedures, particularly open airway surgery including tracheostomy but is applicable to a wide variety of open surgical procedures such as laparotomy. The device protects the surgical team by removing the volume of particulate matter in the environment around the patient.

The evacuator comprises a flexible pad that houses a series of aspiration ports laterally on each side. The ports communicate with outlets that are connected to a high flow, HEPA filtered vacuum system capable of clearing airborne bio-pathogens, including viruses.

The device evacuates air and aerosolized surgical fumes to help reduce viral and other infectious burden in the surgical environment. Without being bound by theory the inclusion of UV light projection within the design of the device can provide another layer of protection to the surgical team.

### Example 3

Cervical Surgical Site Evacuator Embodiment: A Cervical Surgical Site Evacuator can be placed on the surgical field during anterior neck surgery. It is intended for use during aerosol generating procedures, particularly open airway surgery including tracheostomy. The device protects the surgical team by removing the volume of particulate matter in the environment around the patient.

The evacuator can comprise a flexible pad that houses a series of aspiration ports laterally on each side. The ports communicate with outlets that are connected to a high flow, HEPA filtered vacuum system capable of clearing airborne bio-pathogens, including viruses.

### Example 4

The technology involves the use of a suction -evacuation system that conforms to the anatomy of the back of the nose or the nasopharynx and can be introduced either through the mouth or the nose. This device collects plume, secretions and/or aerosolized particles that may be generated during nasal and sinus procedures. The device is meant to be used in a patient who is undergoing a procedure under general anesthesia to reduce risk of infection to the surgeon and operative staff from infectious particles and plume that may be generated during surgical manipulation of the nasal cavity or the nasopharynx.

Description of the uses and commercial applications of the technology: Nasal and sinus procedures are commonly performed procedures for a host of conditions from sinusitis to tumors of the nose and sinuses. The nasal cavity is also the access point for several neurosurgical procedures. Current sy1′ stems and procedures do not allow for collection of blood, secretions, plume or aerosolized infective particles and organisms to be evacuated constantly during the procedure. These bodily products drain form the nose and back of the nose to the throat of the patient while they are asleep. Our device will reduce this exposure of infectious agents and gases to the surgeon and operating room staff and protect the patient from possible aspiration of secretions. The commercial application of this technology is timely since during the current COVID pandemic it has become evident that ENT surgical procedures in the nasal cavity are known to create a significant aerosolization of the viral particles that are concentrated in the nasopharynx, nasal and oral cavity putting the surgical team staff at risk of exposure.

Currently based on our preliminary literature and surgical review, we are not aware of any other device or technology specifically targeting or capable of providing on-going continuous evacuation of secretions and/or plume and aerosolized infectious particles during sinus or endo-nasal procedures while allowing the surgeon to have unobstructed access to both nasal cavities. Our novel concept of placing this device that conforms to and seals the nasopharynx provides the advantage of collecting this material using suction-vacuum technology and also gravity while the patient is in supine position during surgery and also does not interrupt surgical field.

### Example 5

Background: COVID-19, caused by the Severe Acute Respiratory Syndrome-2 virus (SARS-Co-2), has become a pandemic of historic proportion. Being a novel virus, essentially all humans are susceptible, and it is highly contagious. Although most will have mild illness and ultimately recover, many will progress to critical illness; globally, COVID mortality rates are estimated at 5.7% (1). The virus is transmitted primarily through respiratory secretions (2), and is harbored (and shed) in high quantities in the nose, nasopharynx and oral pharynx, even in asymptomatic patients.

To mitigate global spread, the World Health Organization (WHO), Centers for Disease Control and Prevention (CDC), and National Institutes of Health (NIH) have issued guidelines for the general public, including hand-, cough- & sneeze hygiene, social distancing, and self-quarantine, among others. For healthcare workers (HCWs), whose risks are substantially higher, additional recommendations have included use of situation-specific PPE, plus environmental controls. Because the virus is concentrated in the upper respiratory tract (3), risk appears higher for HCWs in Otolaryngology, Anesthesia, Ophthalmology, Pulmonology, and other disciplines where there is need for close-proximity contact with the patient, which results in close-range aerosol transmission (4). Practitioners in these specialties have been among the highest casualties of COVID among HCWs globally. With interventions in the upper airway (e.g., endoscopy, intubation and surgical procedures), the virus is easily aerosolized, further increasing risk of infection among HCWs and support staff members.

In Otolaryngology, many of the upper airway surgeries we perform are classified as Aerosol Generating Procedures (AGPs). Nasal, sinus and skull base surgeries are among the highest AGPs, especially when powered instrumentation such as high-speed drills, shavers and debriders are employed. Even more mundane tools, such as suctions and electrocautery, may contribute to virus aerosolization because of the inherent agitation of secretions and “viral plume” generated with these maneuvers, respectively. In China, the site of the first known outbreak of COVID-19, a single endoscopic pituitary tumor surgery resulted in 14 HCWs infected. Internationally, this has resulted in a drastic decline in upper airway procedures, and authorities have recommended cancellation or postponement of all such elective procedures until further notice (5).

Meanwhile, urgent and emergent conditions still require operative management, e.g., maxillofacial trauma, severe epistaxis, tumors, fulminant infections and others. Surgeons are actively searching for ways of minimizing AGP risk. A variety of suggestions have been promulgated, including use of Powered Air Purifying Respirators, and of negative-pressure ventilation in the operating room (6).

We propose a unique device to help further reduce risk of aerosolization exposures during nasal, sinus and endonasal skull base surgeries. The Nasal Surgical Site Aspirator is a simple device designed to collect, aspirate, and dispose of intraoperative secretions, blood and aerosol.

Device description: This is a surgical site evacuator for trans-nasal procedures. The device will have applications in endoscopic sinus surgery, anterior skull base surgery and facial trauma.

In operation, a trumpet-shaped aspirator is introduced into the nasopharynx at the beginning of surgery. This aspirator is connected to a constant suction (i.e., negative pressure), that has its egress through the mouth. Aspirated secretions and aerosols are continuously evacuated away from the operative field to a sequestered collection device. Key features include the conical-shaped aspirator, a cushion that encircles the aspirator opening and tubing that connects the aspirator to a high-flow, filtered vacuum pump.

We have completed three variations of the design, two for transoral placement and one for positioning through the nasal cavity. Exemplary design specifications are disclosed herein. In each design, the aspirator is connected to the filtered vacuum system by reinforced silicone tubing. For the transoral devices, the cushion can be constructed from silicone coated polyurethane and the aspirator can be constructed from polyvinylchloride plastic. The trans-nasal design can be constructed with medical grade silicone. Cushions could be replaced with inflatable cuffs if needed.

The trans-orally inserted devices are designed to be pressed into position digitally by lifting the soft palate with their leading edge. The soft cushion around the aspirator holds the device in place. The shape of the aspirator allows the surgeon to easily pass the assembly behind the soft palate.

Exemplary device configurations are shown in FIGS. 6A -13B. FIG. 6A illustrates a configuration for trans-oral deployment in the inferior nasopharynx. The design for inferior placement can incorporate openings in the cushion laterally on both sides to avoid a nasopharyngeal vacuum lock and Eustachian tube dysfunction. FIG. 6B shows the inferior nasopharyngeal placement in the context of a lateral airway radiograph.

FIG. 9A illustrates a device design for placement in the superior nasopharynx, posterior to the posterior choanae. The posterior choanal placement will tolerate a full nasopharyngeal seal while evacuating material from the nose (FIG. 9B).

The trans-nasally inserted device is based on the notion that it can be compressed and loaded into an inserter that is passed through the nasal cavity. It requires initial nasal passage of the evacuator tubing that is brought out through the mouth followed by pulling the aspirator into the nasopharynx. Placement would be verified endoscopically. There is no cushion or cuff with this design. The device and inserter are shown in FIGS. 11A and 11B. Preparation for insertion is shown in FIGS. 11C and 11D.

Filtered vacuum pump requirements: For bio-particle filtration systems, the CDC recommends an inlet flow rate of 100 to 150 feet / min (7). This translates in to about 45 m/min for a high flow system. For average nasopharyngeal dimensions, high volumetric flow across the aspirator inlet becomes approximately 14 L/min. Lower flow rates may be appropriate for some procedures. The device will incorporate an inline, disposable, high efficiency particulate air (HEPA) filter.

Other design considerations: The concepts of filtered surgical site evacuation can apply to other upper aerodigestive surgical procedures including oral cavity and peroral endoscopic procedures. Potential variations include placement of a nasopharyngeal evacuator with suction directed toward the oropharynx and an aspirator designed for placement in the oral commissure.

References Cited in This Example
1. Baud et al, Lancet, March 2020.
2. Centers for Disease Control and Prevention. www.cdc.gov/coronavirus/2019-ncov/hcp/guidance-risk-assesment-hcp.html.
3. Zou L, Ruan F, Huang M, et al. SARS-CoV-2 Viral Load in Upper Respiratory Specimens of Infected Patients. N Engl J Med. 2020;382(12):1177-1179.
4. Zara M. Patel, MD;Juan Fernandez-Miranda, MD; Peter H. Hwang, MD; Jayakar V. Nayak, MD, PhD;Robert Dodd, MD, PhD; Hamed Sajjadi, MD;Robert K. Jackler, MD. Precautions For Endoscopic Transnasal Skull Base Surgery During The Covid-19 Pandemic.
5. American Academy of Otolaryngology-Head and Neck Surgery; Specific Nasal Policy, Mar. 19, 2020. www.entnet.org/content/academy-supports-cms-offers-specific-nasal-policy-1
6. Best Practice Recommendations for Pediatric Otolaryngology During the COVID-19 Pandemic. Bann DV, Patel VA, Saadi RA, Goyal N, Gniady JP, McGinn JD, Goldenberg D, Isildak H, May J, Wilson M.; Otolaryngology-Head and Neck Surgery (accepted for publication 2020).
7. Centers for Disease Control and Prevention. Control of smoke from laser/electric surgical procedures. www.cdc.gov/niosh/docs/hazardcontrol/pdfs/hc11.pdf?id=10.26616/NIOSHPUB9612

### Example 6

### A High-Speed Photographic Evaluation of Particle Distribution During Aerosol and Smoke Generating Surgical Procedures: Effects of Surgical Site Evacuation in Exemplary Embodiments

Effectiveness of evacuation systems designed to clear bioaerosols and smoke from the surgical field.

Non-limiting exemplary study design: High-speed photographic evaluation of aerosol and smoke generated in simulated surgical fields.

### Materials and Methods

Surgical site aerosol clearance was evaluated using a model of the anterior neck and prototypes for surgical site evacuator created using 3D printing. A commercially available electrocautery handpiece fitted with an evacuator was tested on animal tissue for smoke clearance. Both systems were connected to a commercial vacuum powered evacuation system. High speed photography was used to record videos of the aerosols and plumes. Fields were recorded with and without evacuation.

Aerosol clearance from an open surgical field using a surgical site evacuator can be dependent upon the suction vent design, airflow velocity, and placement relative to the aerosol generating site. The size and surface geometry of the surgical field can be factors in aerosol clearance.

Surgical smoke generated with electrocautery is cleared from the field by the evacuation enclosure around the handpiece, even at high electrocautery power settings. Except for device noise, there appears to be limited advantage in using evacuator flow rates below the maximum setting.

Bioaerosol and smoke generated during surgery can be sources of respiratory pathogens and pose a threat to operating room personnel. Surgical site evacuation can significantly reduce the volume of airborne particles in the field but requires careful design and deployment considerations.

### Introduction

The COVID-19 pandemic has increased the awareness of pathogenic bioaerosols during aerosol generating procedures (AGPs). The highly contagious SARS-CoV-2 virus can be transmitted through respiratory secretions¹ and concentrated in the nose, nasopharynx, and oral pharynx, even in asymptomatic patients^{2,3}. Health care workers in the operating room who perform procedures associated with high concentrations of the virus in aerosols, such as ear, nose, and throat surgery, can be at risk for infection.

Surgical smoke generated during surgery with heat-producing instruments has been well documented as a risk to exposed operating room personnel. Sources include electrocautery, radiofrequency ablation and laser devices. Surgical smoke is approximately 95% water with the remaining 5% primarily comprising hydrocarbons, tissue particles, viruses, and bacteria⁴. Cultures harvested intraoperatively have documented the presence of live Staphylococcus, Corynebacterium, and Neisseria in surgical plume⁵. Viral pathogens can include HPV and HIV⁶. Toxic / carcinogenic hydrocarbons found in surgical plume can include carbon monoxide, benzene, and hydrogen cyanide⁷.

One direct approach to minimizing the risk of aerosol and smoke producing surgical procedures is disclosed herein - evacuate the particles at the surgical site. We used high speed photography (HSP) to evaluate the effectiveness of a filtered, vacuum powered evacuator to clear aerosols and smoke from the surgical field.

### Materials and Methods

HSP testing platform (see, for example, FIG. 15).

High speed photographic videos were recorded lateral (0 deg) and overhead (90 deg) relative to the plane of the surgical field. The optomechanical equipment to study aerosols and smoke in the surgical field comprised a high-speed camera (Phantom T-1340, Vision Research) fitted with a 100 mm lens (Zeiss Optical), and high-intensity (7700 lumens) LED continuous lighting (Multi-LED, GSVitec). Negative pressure was applied to the evacuators using a surgical smoke evacuation system (Smoke Shark II, Bovie Medical). For optimal field of view, the distal end of the camera lens was placed 46 cm from the center of the surgical field for the lateral view and 58 cm for the overhead view. The LED light source was placed at a 15-degree angle to the camera axis. Images of aerosol and surgical smoke created in the surgical field were recorded at 2000 frames per second with an exposure index of 1600 and a frame exposure time of 10 msec.

High-speed photographic evaluation of surgical site evacuation during aerosol generating procedures.

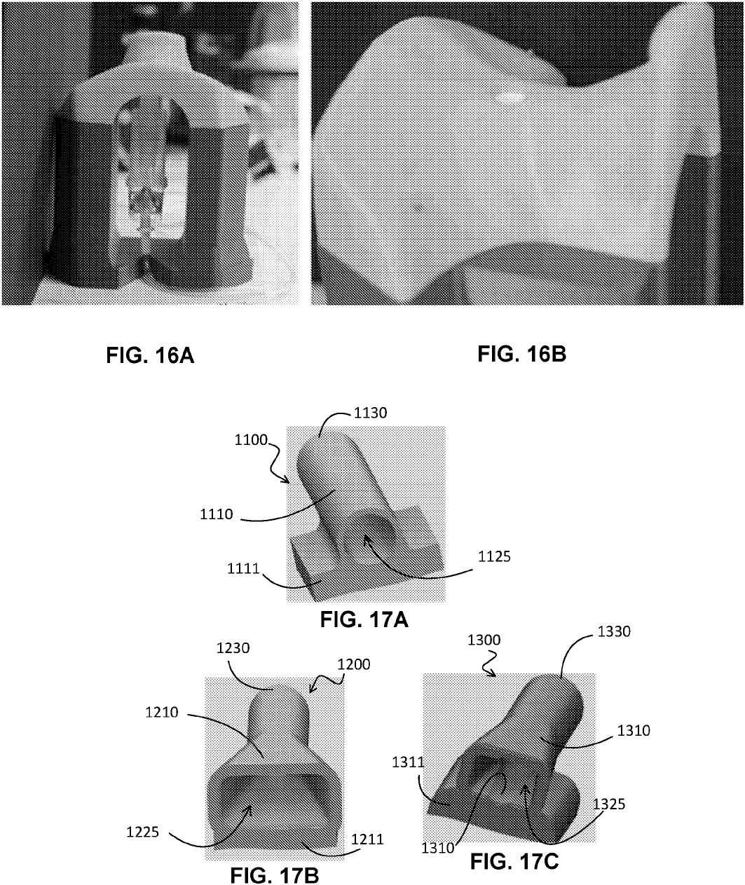

The surgical field of a tracheostomy with an open stoma was simulated using a life-sized manikin of the head and neck (FIG. 16). The anatomical features were 3D printed from a CT scan-generated computer model. An electronic nebulizer (Vios Pro, Pari) was attached to the internal opening of the stoma aperture.

The nebulizer chamber was modified to direct 30% of its flow into the cervical stoma. The remaining flow exited the nebulizer from a port on the side of the chamber and out of the open end of the cervical model, consistent with tracheal airflow. The nebulizer console provided pressured air at a flow rate of 6 L/min, resulting in a stomal airflow of 2 L/min. At the stoma (10 mm diameter), this corresponds to a flow velocity of 0.1 m/s. Assuming a tidal volume of 500 ml/breath and a respiratory rate of 12/min, the nebulizer generated flow is consistent with physiologic flows through the tracheal airway during positive pressure ventilation under general anesthesia.

Three surgical site evacuator designs were tested (FIGS. 17A-17C). The surgical site evacuator was connected to the manikin with adhesive to allow for consistent placement relative to the aerosol. A dual suction vent design was also tested. Surgical site evacuator placement reflected the need to avoid interference with surgical access to the field.

The evacuator suction port was connected to the vacuum console through 9.5 mm or 22 mm corrugated plastic tubing (internal diameter). When the evacuator was set on the maximum setting, the volumetric flow rate was 130 L/min for the 9.5 mm connection and 708 L/min for the 22 mm connection (manufacturer supplied values).

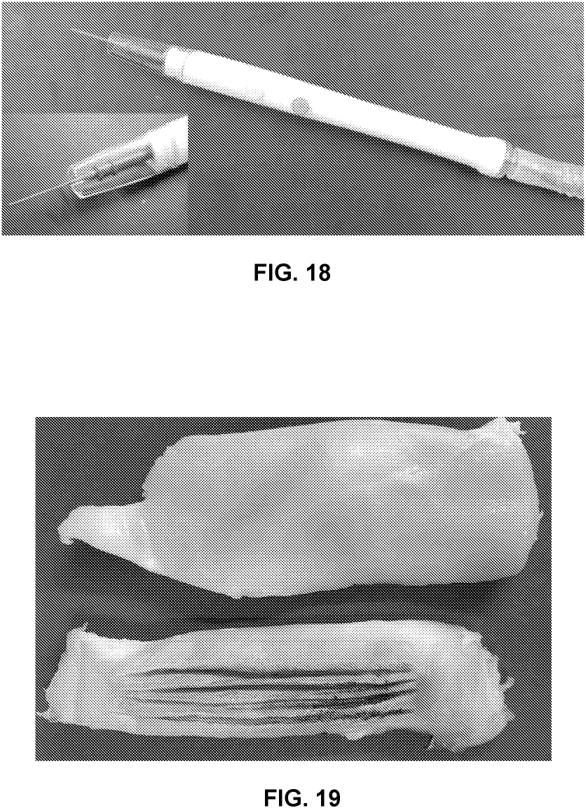

High-speed photographic evaluation of surgical smoke evacuation created by electrocautery. We used a commercial electrosurgical handpiece fitted with a smoke evacuation enclosure (Orca, Bovie Medical) (FIG. 18). The hand piece was connected to its matching electrosurgical console capable of cutting, coagulating and blend frequency settings. The evacuation enclosure was connected to the evacuator through 22.5 mm plastic corrugated tubing. With the evacuator set on the maximum setting, this resulted in volumetric flow of 708 L/ min and a flow velocity of 337 m/s at the enclosure opening. The electrocautery pencil enclosure was also compared to the surgical site evacuators using the same settings.

Skinless chicken breast was used to simulate tissue for electrocautery smoke generation (FIG. 19). A series of electrocautery power and frequency mode settings were tested. The blend setting corresponded to a 25%-on duty cycle.

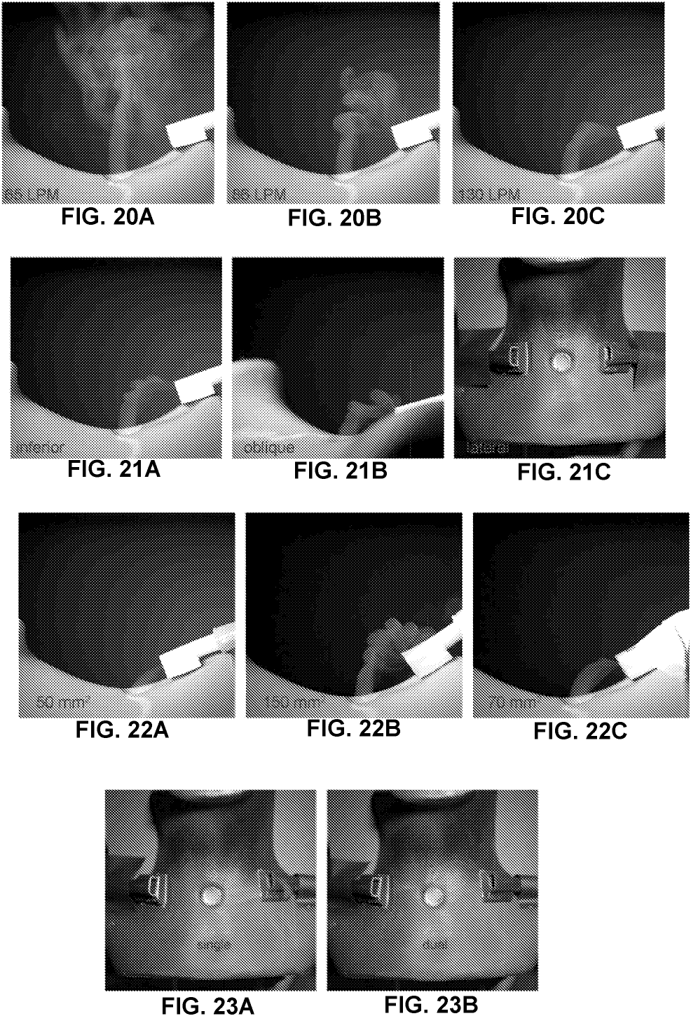

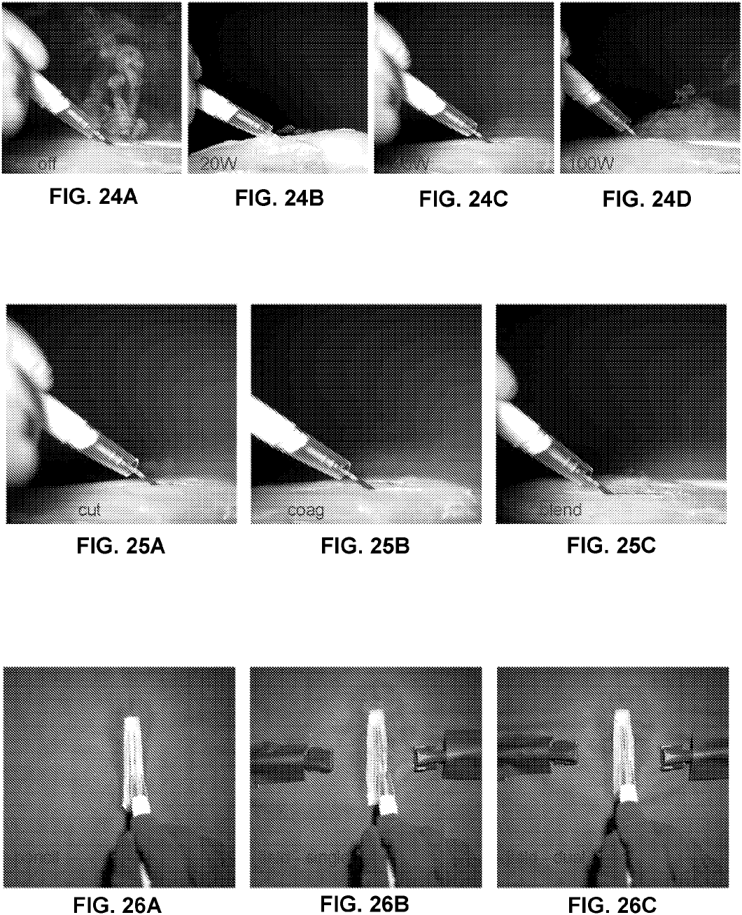

FIGS. 20 through 26 show frame captured images from corresponding videos that were taken during the experimental procedures described herein. The images are grouped to illustrate examples of effects of evacuator parameters on aerosol and smoke clearance. Evacuator effectiveness was rated visually based on the presence of particles traversing the field above the upper plane of the evacuator suction vent opening. Complete evacuation indicates that all particles exiting the stoma or tissue entered the evacuator suction vent; no effect indicates that the surgical evacuator did not appear to alter the trajectory of the particle stream after it entered the field. Partial evacuation indicates that at least some particles escaped evacuation above the field.

Surgical site evacuation during aerosol generating procedures (Table 1).

In some embodiments, the optimal distance from the opening of the evacuator to the center of the stoma can be 3 cm. In the case of cervical anatomy, inferior placement performed better than lateral or oblique placement. This reflects the elevation of the surgical evacuator vent relative to the surgical field. Surgical evacuators with smaller vents performed better than those with larger vents, although the 70 mm² rectangular vent appeared to clear the field as well as the 50 mm² circular vent. Doubling the number of vents resulted in poorer performance at the 130 L/min setting. However, the dual vent system performed well at 708 L/min. These findings can reflect the importance of maintaining an adequate flow velocity at the suction vent opening.

In summary, the 70 mm² rectangular suction vent placed approximately 3 cm from the source at a height of approximately 1 cm above the base of the surgical field appeared to achieve the best site evacuation. Increasing the size of the suction vent or doubling the number of suction vents provides a broader area of field coverage but requires an increase in volumetric flow from the vacuum evacuator to maintain flow velocity. Placing the suction vent below the plane of the source does not clear the field.

TABLE 1

Effectiveness of aerosol evacuation

| | | | |
|---|---|---|---|
| Study | Device (FIG. 3 ) | Parameter | Effectiveness |
| Evac flow | Site evac C | Flow rate - 65 LPM | Partial |
| Evac flow | Site evac C | Flow rate - 86 LPM | Partial |
| Evac flow | Site evac C | Flow rate - 130 LPM | Complete |
| Evac position | Site evac C | Position - inferior, Flow rate 708 LPM | Complete |
| Evac position | Site evac C | Position - oblique, Flow rate 708 LPM | Partial |
| Evac position | Site evac C | Position - lateral, Flow rate 708 LPM | Partial |
| Evac suction vent size | Site evac A | Suction vent opening size - 50 mm², Flow rate 708 LPM | Complete |
| Evac suction vent size | Site evac B | Suction vent opening size - 150 mm², Flow rate 708 LPM | Partial |
| Evac suction vent size | Site evac C | Suction vent opening size - 70 mm², Flow rate 708 LPM | Complete |
| Evac single vs dual | Site evac C | Suction vent number - single, Flow rate 130 LPM | Complete |
| Evac single vs dual | Site evac C | Suction vent number - dual, Flow rate 130 LPM | Partial |
| Evac single vs dual | Site evac C | Suction vent number - single, Flow rate 708 LPM | Complete |
| Evac single vs dual | Site evac C | Suction vent number - dual, Flow rate 708 LPM | Complete |

Smoke evacuation during tissue electrocautery (Table 2).

The electrosurgical pencil enclosure achieved complete evacuation at 20W and 40W power settings, and partial clearance at 100W. Cutting mode produces greater plume volume than coagulation or blend modes but smoke evacuation appeared the same for all modes. For surgical smoke evacuation, the field evacuator can perform at least as well as the pencil enclosure.

TABLE 2

Effectiveness of electrocautery smoke evacuation

| | | | |
|---|---|---|---|
| Study | Device | Parameter | Effectiveness |
| Electrocaut power | Pencil evac | Power - 0 W | No effect |
| Electrocaut power | Pencil evac | Power - 20 W, cut | Complete |
| Electrocaut power | Pencil evac | Power - 40 W, cut | Complete |
| Electrocaut power | Pencil evac | Power - 100 W, cut | Partial |
| Electrocaut mode | Pencil evac | Mode - cut, 40 W | Complete |
| Electrocaut mode | Pencil evac | Mode - coagulate, 40 W | Complete |
| Electrocaut mode | Pencil evac | Mode - blend, 40 W | Complete |
| Electrocaut evac device | Pencil evac | Device - pencil evac, 40 W, cut | Complete |
| Electrocaut evac device | Site evac C | Device - single site evac, 40 W, cut | Complete |
| Electrocaut evac device | Site evac C | Device - dual site evac, 40 W, cut | Complete |

### Discussion

### Surgical Aerosol and Smoke Particle Distributions

Microorganism sizes can vary, depending on the specific microbe. Viruses can be in the about 0.005 - about 0.3 µm (1 micron = 1000 nm) range and bacteria measure around 0.3 - 60 µm. For point of reference, the lower limit of human visual resolution is around 10 µm. A human hair measures around 100 µm in diameter.⁸

Smoke particles can be generated by heat vaporization of tissue, which are approximately 0.1 µm in diameter and rise away from the field with the force of mechanical cellular disruption and heat convection⁹. As they cool, they can form contact aggregates of varying shape with an average (modal) size of approximately 0.2 µm. In contrast, respiratory aerosols, including those generated by surgical instruments, can be the result of high frequency mechanical vibrations. They - 43 -oc be spherical in shape and comprise of water droplets containing cellular material and microorganisms. At their origin, aerosol particles measure about 1 - about 200 µm in diameter. Aerosols can evolve through evaporation and diminish in size with time and distance from their origin. Once the particle sizes diminish to less than 5 µm, they can be considered aerosol nuclei¹⁰. Lightweight aerosol nuclei can be more likely to remain suspended in local air currents where they can be inhaled.

The upper airway, particularly the nose and nasopharynx, traps airborne particles that are greater than about 10 µm in size. Particles reaching the lower respiratory tract can be less than about 7 µm in diameter. Therefore, the most pathogenic aerosolized nuclei particles can be invisible to the naked eye. HEPA filter efficiency ratings vary from about 20% to about 99.5% for particles between about 3 and about10 µm in diameter¹¹. HEPA filters used in surgery can be effective in removing particles down to about 3 µm in size.

Aerosol and smoke detection in the visible light spectrum can be dependent upon the size, concentration, and velocity of the particles or aggregates. Using a photographic system recording 2000 fps with a 100 mm lens (optical 2x magnification) and a resolution of 3270 × 2048 pixels, we can calculate a spatial resolution of approximately 5 µm in the recorded field. Although HSP does not capture the full range of particle sizes in the field, without wishing to be bound by theory, the clearance of the visualized particles is evidence of sufficient evacuation.

### Transmission of Bio-Pathogens in the Operating Room

Three primary models of respiratory pathogen transmission by droplets have been identified¹². Contact transmission can refer to physical contact with respiratory fomites (e.g., hand to mouth or nose). Direct droplet transmission can refer to when pathogen containing liquid droplets are deposited onto respiratory mucus membranes. Airborne transmission can refer to inhalation of air that contains suspended droplet nuclei. For a given pathogen, the transmission modes are not mutually exclusive and spread varies with the local environment in the region of the source.

Smoke generated in the surgical field also can carry bio-pathogens into the local surgical environment around the patient. Smoke differs from respiratory aerosols in that it is the result of tissue vaporization from heat energy. The resulting plume can carry viral particles via convection. Particles are not suspended in liquid droplets and are not propelled by mechanical forces. Additionally, smoke plume can contain harmful hydrocarbons that can contaminate the local environment via diffusion, for example.

There are clinical differences between aerosols and smoke in the operating room. Pathogen laden aerosol droplets can be trapped by surgical personal protective equipment while potentially harmful substances in surgical plume may escape filtration. The significance of hydrocarbons produced from tissue vaporization during surgery can be unclear, but these volatile substances are probably not cleared by available environmental measures to protect healthcare workers during surgery.

### Analysis of Bio-Aerosols in the Surgical Field

Studies of static aerosol distribution have used droplet collection techniques (glass slides, enclosure walls) to estimate the size and number of droplets in the region of an aerosol source¹³. Aerosol mass spectroscopy¹⁴ techniques can be sensitive measures of the composition and quantity of particles suspended in air and can be used in environmental air quality studies. The most used device, the optical particle counter (OPC), is based on particulate scattering of incident light in an enclosed chamber¹⁵. OPC uses a photoelectric sensor that detects the light scattering. The device converts the light reception into a voltage signal that varies directly with the particle size. Except for mass spectroscopy, these aerosol detection methods can be simple and inexpensive to implement. However, they also do not provide any information regarding particle movement over time relative to the aerosol source.

Methods to visualize aerosol dynamics can include high speed photography (HSP)^{16,17,18,19}, light-sheet analysis within a confined chamber^{20,21} or under a microscope¹⁷, and aerosolization of fluorescein solutions²². For direct visualization of particle dynamics, high-resolution HSP can be required. In addition to particle distribution dynamics, HSP has been used to measure particle velocities (particle image velocimetry). Evaluating the effects of surgical evacuation differs from similar studies of aerosol distribution during normally occurring respiratory events (breathing, coughing, sneezing). Most quantitative studies of respiratory droplets focus on the epidemiology of disease transmission²³ where the distribution of potential pathogen bearing particles in the surgical field over time can be more relevant to understanding intraoperative transmission risk.

Studies of naturally occurring respiratory events can be concerned with the distribution of particles as a function of distance from the source, whereas evacuation of the surgical site can be intended to clear the immediate vicinity of the wound. In the case of simulated AGPs, this study documents the effectiveness of droplet clearance within 1 cm from the plane of the surgical wound. Clearance is achieved before the potential droplet cloud can be inhaled by surgical team members, assuming their nose/mouth is approximately 30 cm from the field.

### Protecting Health Care Workers

The primary means of protecting health care workers in the operating room from contaminated aerosol and smoke can be personal protective equipment. For high risk AGPs, high filtration respirators and powered air purifying respirators are recommended. However, these can increase the user’s work of breathing and can lead to operator discomfort, distraction, and fatigue during longer surgical procedures. They can be intrusive and introduce technical surgical limitations, for example, during microscopic, endoscopic, and other minimally invasive procedures. These devices are also subject to cost and availability issues.

Another other means of protection for operating room personnel can be negative pressure ventilation²⁴. This can be designed with other environmental measures such as equipment isolation and limited OR traffic. The effectiveness of negative pressure environments in protecting health care workers has not been documented and there is the possibility that the surgical team is in the path of the bioaerosol flow. Many surgical suites are not equipped with negative pressure ventilation capabilities and retrofitting can be complex and expensive.

Without wishing to be bound by theory, quantitative image processing algorithms can be used to measure particle clearance and optimize the evacuator designs. Particle velocimetry can allow us to determine the extent of flow directional change in the region of the evacuator opening. Using the technical knowledge gained from these experiments, we can complete similar studies of other surgical fields. We have designed embodiments for internal use, such as in trans-nasal and oral cavity procedures (FIG. 6A - FIG. 14). The designs can be suitable for 3D printing and prototyping. HSP techniques learned during this study can allow us to study the production and clearance of aerosols during other AGPs.

High speed photographic imaging of the surgical site documents the use for evacuation of aerosols and plumes produced by aerosol generating procedures and smoke producing surgical devices. Effective surgical site aerosol evacuation requires careful design of the surgical evacuator and proper placement in the field. The commercially available evacuation enclosure used in this study is effective for electrocautery smoke clearance.

### REFERENCES CITED IN THIS EXAMPLE

1. Center for Disease Control: Interim U.S. Guidance for Risk Assessment and Public Health Management of Healthcare Personnel with Potential Exposure in a Healthcare Setting to Patients with Coronavirus Disease 2019 (COVID-19). https://www.cdc.gov/coronavirus/2019-ncov/hcp/guidance-risk-assesment-hcp.html. Accessed May 10, 2020.

2. Pan, Y., et al., Viral load of SARS-CoV-2 in clinical samples. Lancet Infect Dis, 2020. 20(4): p. 411-412.

3. Zou, L., et al., SARS-CoV-2 Viral Load in Upper Respiratory Specimens of Infected Patients. N Engl J Med, 2020. 382(12): p. 1177-1179.

4. Gonzalez-Bayon L, Gonzalez-Moreno S, Ortega-Perez G. Safety considerations for operating room personnel during hyperthermic intraoperative intraperitoneal chemotherapy perfusion. Eur J Surg Oncol. 2006;32(6):619-624.

5. Capizzi PJ, Clay RP, Battey MJ. Microbiologic activity in laser resurfacing plume and debris. Lasers Surg Med. 1998;23(3):172-4.

6. Garden JM, O’Banion MK, Bakus AD, Olson C. Viral disease transmitted by laser-generated plume (aerosol). Arch Dermatol. 2002; 138: 1303-1307.

7. Choi SH, Kwon TG, Chung SK, Kim, TH. Surgical smoke may be a biohazard to surgeons performing laparoscopic surgery. Surg Endosc. 2014;28:2374-2380.

8. Staley JT. Bacteria, Their Smallest Representatives and Subcellular Structures, and the Purported Precambrian Fossil “Metallogenium”. in Size Limits of Very Small Microorganisms: Proceedings of a Workshop. 1999. National Academies Press.

9. Qiao, Y, Andrews AJ, Christen CE, et al. Morphological characterization of particles emitted from monopolar electrosurgical pencils. Journal of Aerosol Science. 2020;142: 105512.

10. Fennelly, KP. Particle sizes of infectious aerosols: implications for infection control. The Lancet. Respiratory medicine. 2020;8(9):914-924.

11. Christopherson DA, Yao WC, Lu M, et al. High-efficiency particulate air filters in the era of COVID-19: function and efficacy. Otolaryngology-Head and Neck Surgery. 2020;163(6):1153-1155.

12. Tellier R. Review of aerosol transmission of influenza A in human beings. Lancet Infect Dis. 2006;20:1657-1662.

13. Xie X, Li Y, Sun H, Liu L. Exhaled droplets due to talking and coughing. J. R. Soc. Interface. 2009;6:S703-S714.

14. Nash DG, Baer T, Johnston MV. Aerosol mass spectrometry: An introductory review. International Journal of Mass Spectrometry. 2006;258:2-12.

15. Simpson JP, Wong DN, Verco L, et al. Measurement of airborne particle exposure during simulated tracheal intubation using various proposed aerosol containment devices during the COVID-19 pandemic. Anaesthesia. 2020;75(12):1587-1595.

16. Bourouiba, L., Images in clinical medicine. A sneeze. N Engl J Med, 2016. 375(8):e15.

17. Zhao, H., et al., Simultaneous measurement of velocities and size distribution of fine atmospheric aerosols based on image processing and PTV techniques. Atmospheric Environment, 2005. 39(17):3015-3021.

18. Tang, J.W., et al., Airflow dynamics of human jets: sneezing and breathing-potential sources of infectious aerosols. PLoS One, 2013. 8(4).

19. Nishimura, H., S. Sakata, and A. Kaga, A new methodology for studying dynamics of aerosol particles in sneeze and cough using a digital high-vision, high-speed video system and vector analyses. PloS one, 2013. 8(11).

20. Stadnytskyi, V., et al., The airborne lifetime of small speech droplets and their potential importance in SARS-CoV-2 transmission. Proceedings of the National Academy of Sciences, 2020: p. 202006874.

21. McLean, J.A., et al., Optical patternation: a technique for three-dimensional aerosol diagnostics. Analytical chemistry, 2000. 72(20): p. 4796-4804.

22. Workman, A.D., et al., Endonasal instrumentation and aerosolization risk in the era of COVID-19: simulation, literature review, and proposed mitigation strategies. Int Forum Allergy Rhinol, 2020.

23. Stilianakis NI, Drossinos Y. Dynamics of infectious disease transmission by inhalable respiratory droplets. J. R. Soc. Interface. 2010;7:1355-1366.

24. Ti, L.K., et al., What we do when a COVID-19 patient needs an operation: operating room preparation and guidance. Can J Anaesth, 2020. 67(6): p. 756-758.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A surgical evacuator comprising:
a suction port, a body, at least one suction vent,
the suction port being in fluid communication with the body; and
the body being in fluid communication with the at least one suction vent, wherein the surgical evacuator is configured to remove surgical debris that can be created during a surgical procedure.

2. The surgical evacuator of claim 1, further comprising a HEPA filtered vacuum system.

3. The surgical evacuator of claim 1, wherein the surgical debris comprises surgical plume, surgical fumes, intraoperative secretions, blood, infectious aerosols, or a combination thereof.

4. The surgical evacuator of claim 1, further comprising an ultraviolet light configured to eradicate microorganisms and viruses.

5. The surgical evacuator of claim 1, comprising two suction ports.

6. The surgical evacuator of claim 1, wherein the suction port comprises a tubular extension extending from an external surface of the body, and the suction port is configured to reversibly attach a tubing such that the suction port is in fluid communication with the tubing.

7. The surgical evacuator of claim 6, further comprising a valve to selectively control fluid communication between the suction port and the tubing.

8. The surgical evacuator of claim 6, wherein the suction port further comprises a Luer-lock or Luer-taper hub.

9. The surgical evacuator of claim 6, wherein the suction port is configured to frictionally attach the tubing thereto.

10. The surgical evacuator of claim 1, wherein the surgical evacuator comprises a medical grade metal, a medical-grade polymer, or a combination thereof.

11. The surgical evacuator of claim 10, wherein the medical grade metal comprises stainless steel, titanium, tantalum, gold, platinum, palladium, or a combination thereof.

12. The surgical evacuator of claim 10, wherein the surgical evacuator comprises a silicone elastomer, sterilizable plastic, polytetrafluoroethylene, polyether block amide, polyvinyl chloride, or a combination thereof.

13. The surgical evacuator of claim 1, wherein the surgical evacuator is reusable.

14. The surgical evacuator of claim 1, wherein the surgical evacuator is disposable..

15. The surgical evacuator of claim 1, wherein the body comprises a manifold and the manifold comprises a plurality of suction vents.

16. The surgical evacuator of claim 15, comprising at least three suction vents.

17. The surgical evacuator of claim 15, wherein the plurality of suction vents are configured to at least partially surround a surgical field.

18. The surgical evacuator of claim 17, wherein the surgical evacuator is configured for use in open surgical procedures.

19. The surgical evacuator of claim 18, wherein the surgical evacuator is configured for use with procedures that comprise an open wound.

20. The surgical evacuator of claim 18, wherein the surgical evacuator is configured for use in a tracheostomy procedure, an otological procedure, a laparotomy, or a combination thereof.

21. The surgical evacuator of claim 17, wherein the surgical evacuator is configured to be placed over a surgical drape.

22. The surgical evacuator of claim 17, wherein the surgical evacuator is configured to reside within an opening of a surgical drape.

23. The surgical evacuator of claim 17, wherein the surgical field comprises an open wound.

24. The surgical evacuator of claim 17, wherein the surgical field comprises an otolaryngolical surgical field or an abdominal surgical field.

25. The surgical evacuator of claim 17, wherein the surgical evacuator is configured to reside upon a lateral skull of a patient and surround an ear of the patient.

26. The surgical evacuator of claim 25, wherein the surgical evacuator comprises a C-shaped design.

27. The surgical evacuator of claim 25, wherein the surgical evacuator comprises a substantially circular or substantially rectangular design.

28. The surgical evacuator of claim 17, wherein the surgical evacuator is configured to reside upon the ventral side of a neck of a patient.

29. The surgical evacuator of claim 28, wherein the surgical evacuator is configured for use in an open airway surgery.

30. The surgical evacuator of claim 29, wherein the surgical evacuator is configured for use in a tracheostomy procedure.

31. The surgical evacuator of claim 28, wherein the surgical evacuator comprises an arched rectangular design or an arched elliptical design.

32. The surgical evacuator of claim 31, comprising at least two manifolds, at least two suction ports, and at least two pluralities of suction vents,
wherein a first manifold is in fluid communication with a first suction port and a first plurality of suction vents, and
a second manifold is in fluid communication with a second suction port and a second plurality of suction vents.

33. The surgical evacuator of claim 32, wherein the first manifold and second manifold are disposed on opposite sides of the rectangular design.

34. The surgical evacuator of claim 17, wherein the surgical evacuator is flexible.

35. The surgical evacuator of claim 6, wherein the surgical evacuator is configured for use with nasal procedures, nasopharyngeal procedures, upper aerodigestive procedures, dental procedures, or a combination thereof; and
the surgical evacuator is configured for transoral or trans-nasal placement.

36. The surgical evacuator of claim 35, wherein upper aerodigestive procedures comprise oral cavity procedures, peroral endoscopic procedures, or a combination thereof.

37. The surgical evacuator of claim 35, wherein the surgical evacuator is configured for use in nasal surgery, sinus surgery, endonasal skull base surgery, anterior skull base surgery, facial trauma surgery or a combination thereof.

38. The surgical evacuator of claim 35, wherein the body comprises an aspirator, and the at least one suction vent comprises an opening of the aspirator.

39. The surgical evacuator of claim 38, wherein the surgical evacuator is configured to conform anatomy of the back of a patient’s nose or a patient’s nasopharynx.

40. The surgical evacuator of claim 38, wherein the aspirator comprises a substantially conical shape or a substantially cylindrical shape.

41. The surgical evacuator of claim 38, further comprising a cushion that encircles the opening of the aspirator.

42. The surgical evacuator of claim 41, wherein the cushion comprises any pliable-or semi-pliable medical-grade material.

43. The surgical evacuator of claim 41, wherein the cushion comprises a soft rubber-like material.

44. The surgical evacuator of claim 41, wherein the cushion comprises a silicone-coated polyurethane material.

45. The surgical evacuator of claim 38, further comprising an inflatable cuff that encircles the opening of the aspirator.

46. The surgical evacuator of claim 38, wherein the surgical evacuator is configured to direct suction toward a patient’s oropharynx.

47. The surgical evacuator of claim 38, wherein the surgical evacuator is configured for placement in a patient’s oral commissure.

48. The surgical evacuator of claim 38, wherein the surgical evacuator is configured for inferior nasopharyngeal placement, superior nasopharyngeal, or a combination thereof.

49. The surgical evacuator of claim 48, wherein the aspirator comprises a means for avoiding a nasopharyngeal vacuum lock.

50. The surgical evacuator of claim 49, wherein the means for avoiding a nasopharyngeal vacuum lock comprises at least one gap, hole, or channel along a perimeter of the aspirator opening.

51. The surgical evacuator of claim 49, further comprising a cushion or inflatable cuff that encircles the opening of the aspirator, and the means for avoiding a nasopharyngeal vacuum lock comprises at least one gap, hole, or channel that is disposed upon or within the cushion or inflatable cuff.

52. The surgical evacuator of claim 38, further comprising an inserter that is configured for use with trans-nasal placement of the evacuator, wherein the inserter is configured to receive and hold the surgical evacuator.

53. A surgical evacuator comprising:
a suction port, a manifold, and a plurality of suction vents, wherein the plurality of suction vents at least partially surrounds a surgical field;
the surgical port being in fluid communication with the manifold;
the manifold being in fluid communication with the plurality of vents; and
the surgical evacuator being configured to remove surgical debris during an open surgical procedure.

54. The surgical evacuator of claim 53, wherein the surgical debris comprises surgical plume, intraoperative secretions, blood, infectious aerosols, or a combination thereof.

55. The surgical evacuator of claim 53 comprising at least three suction vents.

56. The surgical evacuator of claim 55 comprising at least five suction vents.

57. The surgical evacuator of claim 56 comprising at least six suction vents.

58. A surgical evacuator comprising:
a suction port, a body, and at least one suction vent, wherein the body comprises an aspirator, and the at least one suction vent comprises an opening of the aspirator;
the suction port being in fluid communication with the aspirator; and
the suction port comprising a tubular extension extending from an external surface of the body, and the suction port being configured to reversibly attach a tubing to permit fluid communication between the suction port and the tubing;
the body being in fluid communication with the at least one suction vent; and
the surgical evacuator being configured to remove surgical debris during a nasal or nasopharyngeal surgical procedure.

59. The surgical evacuator of claim 58, wherein the surgical debris comprises surgical plume, intraoperative secretions, blood, infectious aerosols, or a combination thereof.

60. A method of removing aerosolized surgical debris during a surgical procedure, the method comprising:
obtaining the surgical evacuator of any one of claims 1-34;
placing the surgical evacuator around at least a portion of the surgical field;
attaching the suction port to a suction system, wherein the suction system comprises a tubing and a HEPA filtered vacuum system;
activating the suction system to generate a vacuum within the surgical port, the body, and the at least one suction vent; and
permitting aerosolized surgical debris to be drawn into the surgical evacuator serially through the at least one suction vent, the body, and exit through the suction port to the tubing for disposal within the HEPA filtered vacuum system.

61. A method of removing aerosolized surgical debris during a surgical procedure, the method comprising:
obtaining the surgical evacuator of any one of claims 53-57;
placing the surgical evacuator around at least a portion of the surgical field;
attaching the suction port to a suction system, wherein the suction system comprises a tubing and a HEPA filtered vacuum system;
activating the suction system to generate a vacuum within the surgical port, the manifold, and the plurality of suction vents; and
permitting aerosolized surgical debris to be drawn into the surgical evacuator serially through the plurality of suction vents, the manifold, and then exit through the suction port to the tubing for disposal within the HEPA filtered vacuum system.

62. A method of removing aerosolized surgical debris during a surgical procedure, the method comprising:
obtaining the surgical evacuator of any one of claims 1-16 or 35-52;
placing the surgical evacuator within a patient’s inferior nasopharynx or within a patient’s superior nasopharynx;
attaching the suction port to a suction system, wherein the suction system comprises a tubing and a HEPA filtered vacuum system;
activating the suction system to generate a vacuum within the surgical port, the body, and the at least one suction vent; and
permitting aerosolized surgical debris to be drawn into the surgical evacuator serially through the at least one suction vent, the body, and exit through the suction port to the tubing for disposal within the HEPA filtered vacuum system.

63. The method of claim 62, further comprising an inserter that is configured to receive and hold the surgical evacuator and evacuator tubing; the method comprising:
placing the inserter into the nasal cavity and loading the surgical evacuator into the inserter;
wherein the step of placing the surgical evacuator comprises:
passing the evacuator tubing through the patient’s nasal cavity and into the patient’s oral cavity;
bringing the tubing out through the patient’s mouth;
pulling tubing to draw the inserter and the surgical evacuator into a nasal passage of the patient; and
further pulling of the tubing to draw the surgical evacuator out of the inserter and into the patient’s nasopharynx.

64. The method of claim 63, further comprising endoscopically verifying placement of the surgical evacuator.

65. A method of reducing healthcare provider exposure to infectious aerosolized surgical debris during a surgical procedure, the method comprising:
obtaining the surgical evacuator of any one of claims 1-34;
placing the surgical evacuator around at least a portion of the surgical field;
attaching the suction port to a suction system, wherein the suction system comprises a tubing and a HEPA filtered vacuum system;
activating the suction system to generate a vacuum within the surgical port, the body, and the at least one suction vent; and
capturing infectious aerosolized surgical debris through the surgical evacuator before the infectious aerosolized surgical debris can leave the surgical field, thereby reducing healthcare provider exposure to the infectious surgical debris.

66. A method of reducing healthcare provider exposure to infectious aerosolized surgical debris during a surgical procedure, the method comprising:
obtaining the surgical evacuator of any one of claims 53-57;
placing the surgical evacuator around at least a portion of the surgical field;
attaching the suction port to a suction system, wherein the suction system comprises a tubing and a HEPA filtered vacuum system;
activating the suction system to generate a vacuum within the surgical port, the manifold, and the plurality of suction vents; and
capturing infectious aerosolized surgical debris through the surgical evacuator before the infectious aerosolized surgical debris can leave the surgical field, thereby reducing healthcare provider exposure to the infectious surgical debris.

67. A method of reducing healthcare provider exposure to infectious aerosolized surgical debris during a surgical procedure, the method comprising:
obtaining the surgical evacuator of any one of claims 1-16 or 35-52;
placing the surgical evacuator within a patient’s inferior nasopharynx or within a patient’s superior nasopharynx;
attaching the suction port to a suction system, wherein the suction system comprises a tubing and a HEPA filtered vacuum system;
activating the suction system to generate a vacuum within the surgical port, the body, and the at least one suction vent; and
capturing infectious aerosolized surgical debris through the surgical evacuator before the infectious aerosolized surgical debris can leave the surgical field, thereby reducing healthcare provider exposure to the infectious surgical debris.

68. The method of claim 67, further comprising an inserter that is configured to receive and hold the surgical evacuator and evacuator tubing; the method comprising:
loading the surgical evacuator into the inserter;
wherein the step of placing the surgical evacuator comprises:
passing the evacuator tubing through the patient’s nasal cavity and into the patient’s oral cavity;
bringing the tubing out through the patient’s mouth;
pulling tubing to draw the inserter and the surgical evacuator into a nasal passage of the patient; and
further pulling of the tubing to draw the surgical evacuator out of the inserter and into the patient’s nasopharynx.

69. The method of claim 67, further comprising endoscopically verifying placement of the surgical evacuator.
